SEQUENCE LISTING (10 sequences)

SEQ ID NO: 1 - DNA, 6524 bp, Rattus norvegicus
  CDS (204)..(6074)
tagcttgcttctgctaatgctaccccaggcctttagacagagaacagatggcagatggagtttcttattgccatgcgcaaacgctgagcccacctcatgatcccggaccccatggttttcagtagacaacctgggctaagaagagatctccgaccttatagagcagcaaagagtgtaaattcttccccaagaagaatgagaagatggagctcccctttgcgtccgtgggaactaccaatttcagacggttcactccagagtcactggcagagatcgagaagcagattgctgctcaccgcgcagccaagaaggccagaaccaagcacagaggacaggaggacaagggcgagaagcccaggcctcagctggacttgaaagactgtaaccagctgcccaagttctatggtgagctcccagcagaactggtcggggagcccctggaggacctagaccctttctacagcacacaccggacattcatggtgttgaataaaagcaggaccatttccagattcagtgccacttgggccctgtggctcttcagtcccttcaacctgatcagaagaacagccatcaaagtgtctgtccattcctggttctccatattcatcaccatcactattttggtcaactgcgtgtgcatgacccgaactgatcttccagagaaagtcgagtacgtcttcactgtcatttacaccttcgaggctctgattaagatactggcaagagggttttgtctaaatgagttcacttatcttcgagatccgtggaactggctggacttcagtgtcattaccttggcgtatgtgggtgcagcgatagacctccgaggaatctcaggcctgcggacattccgagttctcagagccctgaaaactgtttctgtgatcccaggactgaaggtcatcgtgggagccctgatccactcagtgaggaagctggccgacgtgactatcctcacagtcttctgcctgagcgtcttcgccttggtgggcctgcagctctttaaggggaaccttaagaacaaatgcatcaggaacggaacagatccccacaaggctgacaacctctcatctgaaatggcagaatacatcttcatcaagcctggtactacggatcccttactgtgcggcaatgggtctgatgctggtcactgccctggaggctatgtctgcctgaaaactcctgacaacccggattttaactacaccagctttgattcctttgcgtgggcattcctctcactgttccgcctcatgacgcaggactcctgggagcgcctgtaccagcagacactccgggcttctgggaaaatgtacatggtctttttcgtgctggttattttccttggatcgttctacctggtcaatttgatcttggccgtggtcaccatggcgtatgaagagcagagccaggcaacaattgcagaaatcgaagccaaggaaaaaaagttccaggaagcccttgaggtgctgcagaaggaacaggaggtgctggaagccctggggattgacacgacctcgctccagtcccacagtggatcacccttagcctccaaaaacgccaatgagagaagacccagggtgaaatcaagggtgtcagagggctccacggatgacaacaggtcaccccaatctgacccttacaaccagcgcaggatgtctttcctaggcctgtcttcaggaagacgcagggctagccacggcagtgtgttccacttccgagcgcccagccaagacatctcatttcctgacgggatcacccctgatgatggggtctttcacggagaccaggaaagccgtcgaggttccatattgctgggcaggggtgctgggcagacaggtccactccccaggagcccactgcctcagtcccccaaccctggccgtagacatggagaagagggacagctcggagtgcccactggtgagcttaccgctggagcgcctgaaggcccggcactgcacactacagggcagaagagcttcctgtctgcgggctacttgaacgaacctttccgagcacagagggccatgagcgttgtcagtatcatgacttctgtcattgaggagcttgaagagtctaagctgaagtgcccaccctgcttgatcagcttcgctcagaagtatctgatctgggagtgctgccccaagtggaggaagttcaagatggcgctgttcgagctggtgactgaccccttcgcagagcttaccatcaccctctgcatcgtggtgaacaccgtcttcatggccatggagcactaccccatgaccgatgccttcgatgccatgcttcaagccggcaacattgtcttcaccgtgtttttcacaatggagatggccttcaagatcattgccttcgacccctactattacttccagaagaagtggaatatcttcgactgtgtcatcgtcaccgtgagccttctggagctgagtgcatccaagaagggcagcctgtctgtgctccgtaccttacgcttgctgcgggtcttcaagctggccaagtcctggcccaccctgaacaccctcatcaagatcatcgggaactcagtgggggccctgggcaacctgacctttatcctggccatcatcgtcttcatcttcgccctggtcggaaagcagcttctctcagaggactacgggtgccgcaaggacggcgtctccgtgtggaacggcgagaagctccgctggcacatgtgtgacttcttccattccttcctggtcgtcttccgaatcctctgcggggagtggatcgagaacatgtgggtctgcatggaggtcagccagaaatccatctgcctcatcctcttcttgactgtgatggtgctgggcaacctagtggtgctcaaccttttcatcgctttactgctgaactccttcagcgcggacaacctcacggctccagaggatgacggggaggtgaacaacttgcagttagcactggccaggatccaggtacttggccatcgggccagcagggccatcgccagttacatcagcagccactgccgattccactggcccaaggtggagacccagctgggcatgaagcccccactcaccagctcagaggccaagaaccacattgccactgatgctgtcagtgctgcagtggggaacctgacaaagccagctctcagtagccccaaggagaaccacggggacttcatcactgatcccaacgtgtgggtctctgtgcccattgctgagggggaatctgacctcgacgagctcgaggaagatatggagcaggcttcgcagagctcctggcaggaagaggaccccaagggacagcaggagcagttgccacaagtccaaaagtgtgaaaaccaccaggcagccagaagcccagcctccatgatgtcctctgaggacctggctccatacctgggtgagagctggaagaggaaggatagccctcaggtccctgccgagggagtggatgacacgagctcctctgagggcagcacggtggactgcccggacccagaggaaatcctgaggaagatccccgagctggcagatgacctggacgagcccgatgactgtttcacagaaggctgcactcgccgctgtccctgctgcaacgtgaatactagcaagtctccttgggccacaggctggcaggtgcgcaagacctgctaccgcatcgtggagcacagctggtttgagagtttcatcatcttcatgatcctgctcagcagtggagcgctggcctttgaggataactacctggaagagaaaccccgagtgaagtccgtgctggagtacactgaccgagtgttcaccttcatcttcgtctttgagatgctgctcaagtgggtagcctatggcttcaaaaagtatttcaccaatgcctggtgctggctggacttcctcattgtgaacatctccctgacaagcctcatagcgaagatccttgagtattccgacgtggcgtccatcaaagcccttcggactctccgtgccctccgaccgctgcgggctctgtctcgattcgaaggcatgagggtagtggtggatgccctcgtgggcgccatcccctccatcatgaacgtcctcctcgtctgcctcatcttctggctcatcttcagcatcatgggcgtgaacctcttcgccgggaaattttcgaagtgcgtcgacaccagaaataacccattttccaacgtgaattcgacgatggtgaataacaagtccgagtgtcacaatcaaaacagcaccggccacttcttctgggtcaacgtcaaagtcaacttcgacaacgtcgctatgggctacctcgcacttcttcaggtggcaaccttcaaaggctggatggacataatgtatgcagctgttgattccggagagatcaacagtcagcctaactgggagaacaacttgtacatgtacctgtacttcgtcgttttcatcattttcggtggcttcttcacgctgaatctctttgttggggtcataatcgacaacttcaaccaacagaaaaaaaagctaggaggccaggacatcttcatgacagaagagcagaagaagtactacaatgccatgaagaagctgggctccaagaaaccccagaagcccatcccacggcccctgaataagtaccaaggcttcgtgtttgacatcgtgaccaggcaagcctttgacatcatcatcatggttctcatctgcctcaacatgatcaccatgatggtggagaccgacgagcagggcgaggagaagacgaaggttctgggcagaatcaaccagttctttgtggccgtcttcacgggcgagtgtgtgatgaagatgttcgccctgcgacagtactacttcaccaacggctggaacgtgttcgacttcatagtggtgatcctgtccattgggagtctgctgttttctgcaatccttaagtcactggaaaactacttctccccgacgctcttccgggtcatccgtctggccaggatcggccgcatcctcaggctgatccgagcagccaaggggattcgcacgctgctcttcgccctcatgatgtccctgcccgccctcttcaacatcggcctcctcctcttcctcgtcatgttcatctactccatcttcggcatggccagcttcgctaacgtcgtggacgaggccggcatcgacgacatgttcaacttcaagacctttggcaacagcatgctgtgcctgttccagatcaccacctcggccggctgggacggcctcctcagccccatcctcaacacggggcctccctactgcgaccccaacctgcccaacagcaacggctcccgggggaactgcgggagcccggcggtgggcatcatcttcttcaccacctacatcatcatctccttcctcatcgtggtcaacatgtacatcgcagtgattctggagaacttcaacgtagccaccgaggagagcacggagcccctgagcgaggacgacttcgacatgttctatgagacctgggagaagttcgacccggaggccacccagttcattgccttttctgccctctcagacttcgcggacacgctctccggccctcttagaatccccaaacccaaccagaatatattaatccagatggacctgccgttggtccccggggataagatccactgtctggacatcctttttgccttcacaaagaacgtcttgggagaatccggggagttggactccctgaagaccaatatggaagagaagtttatggcgaccaatctctccaaagcatcctatgaaccaatagccaccaccctccggtggaagcaggaagacctctcagccacagtcattcaaaaggcctaccggagctacatgctgcaccgctccttgacactctccaacaccctgcatgtgcccagggctgaggaggatggcgtgtcacttcccggggaaggctacattacattcatggcaaacagtggactcccggacaaatcagaaactgcctctgctacgtctttcccgccatcctatgacagtgtcaccaggggcctgagtgaccgggccaacattaacccatctagctcaatgcaaaatgaagatgaggtcgctgctaaggaaggaaacagccctggacctcagtgaaggcactcaggcatgcacagggcaggttccaatgtctttctctgctgtactaactccttccctctggaggtggcaccaacctccagcctccaccaatgcatgtcactggtcatggtgtcagaactgaatggggacatccttgagaaagcccccaccccaataggaatcaaaagccaaggatactcctccattctgacgtcccttccgagttcccagaagatgtcattgctcccttctgtttgtgaccagagacgtgattcaccaacttctcggagccagagacacatagcaaagacttttctgctggtgtcgggcagtcttagagaagtcacgtaggggttggtactgagaattagggtttgcatgactgcatgctcacagctgccggacaatacctgtgagtcggccattaaaattaatatttttaaagttaaaaaaaaaaaaaaa

SEQ ID NO: 2 - protein, 1957 aa, Rattus norvegicus
MELPFASVGTTNFRRFTPESLAEIEKQIAAHRAAKKARTKHRGQEDKGEKPRPQLDLKDCNQLPKFYGELPAELVGEPLEDLDPFYSTHRTFMVLNKSRTISRFSATWALWLFSPFNLIRRTAIKVSVHSWFSIFITITILVNCVCMTRTDLPEKVEYVFTVIYTFEALIKILARGFCLNEFTYLRDPWNWLDFSVITLAYVGAAIDLRGISGLRTFRVLRALKTVSVIPGLKVIVGALIHSVRKLADVTILTVFCLSVFALVGLQLFKGNLKNKCIRNGTDPHKADNLSSEMAEYIFIKPGTTDPLLCGNGSDAGHCPGGYVCLKTPDNPDFNYTSFDSFAWAFLSLFRLMTQDSWERLYQQTLRASGKMYMVFFVLVIFLGSFYLVNLILAVVTMAYEEQSQATIAEIEAKEKKFQEALEVLQKEQEVLEALGIDTTSLQSHSGSPLASKNANERRPRVKSRVSEGSTDDNRSPQSDPYNQRRMSFLGLSSGRRRASHGSVFHFRAPSQDISFPDGITPDDGVFHGDQESRRGSILLGRGAGQTGPLPRSPLPQSPNPGRRHGEEGQLGVPTGELTAGAPEGPALHTTGQKSFLSAGYLNEPFRAQRAMSVVSIMTSVIEELEESKLKCPPCLISFAQKYLIWECCPKWRKFKMALFELVTDPFAELTITLCIVVNTVFMAMEHYPMTDAFDAMLQAGNIVFTVFFTMEMAFKIIAFDPYYYFQKKWNIFDCVIVTVSLLELSASKKGSLSVLRTLRLLRVFKLAKSWPTLNTLIKIIGNSVGALGNLTFILAIIVFIFALVGKQLLSEDYGCRKDGVSVWNGEKLRWHMCDFFHSFLVVFRILCGEWIENMWVCMEVSQKSICLILFLTVMVLGNLVVLNLFIALLLNSFSADNLTAPEDDGEVNNLQLALARIQVLGHRASRAIASYISSHCRFHWPKVETQLGMKPPLTSSEAKNHIATDAVSAAVGNLTKPALSSPKENHGDFITDPNVWVSVPIAEGESDLDELEEDMEQASQSSWQEEDPKGQQEQLPQVQKCENHQAARSPASMMSSEDLAPYLGESWKRKDSPQVPAEGVDDTSSSEGSTVDCPDPEEILRKIPELADDLDEPDDCFTEGCTRRCPCCNVNTSKSPWATGWQVRKTCYRIVEHSWFESFIIFMILLSSGALAFEDNYLEEKPRVKSVLEYTDRVFTFIFVFEMLLKWVAYGFKKYFTNAWCWLDFLIVNISLTSLIAKILEYSDVASIKALRTLRALRPLRALSRFEGMRVVVDALVGAIPSIMNVLLVCLIFWLIFSIMGVNLFAGKFSKCVDTRNNPFSNVNSTMVNNKSECHNQNSTGHFFWVNVKVNFDNVAMGYLALLQVATFKGWMDIMYAAVDSGEINSQPNWENNLYMYLYFVVFIIFGGFFTLNLFVGVIIDNFNQQKKKLGGQDIFMTEEQKKYYNAMKKLGSKKPQKPIPRPLNKYQGFVFDIVTRQAFDIIIMVLICLNMITMMVETDEQGEEKTKVLGRINQFFVAVFTGECVMKMFALRQYYFTNGWNVFDFIVVILSIGSLLFSAILKSLENYFSPTLFRVIRLARIGRILRLIRAAKGIRTLLFALMMSLPALFNIGLLLFLVMFIYSIFGMASFANVVDEAGIDDMFNFKTFGNSMLCLFQITTSAGWDGLLSPILNTGPPYCDPNLPNSNGSRGNCGSPAVGIIFFTTYIIISFLIVVNMYIAVILENFNVATEESTEPLSEDDFDMFYETWEKFDPEATQFIAFSALSDFADTLSGPLRIPKPNQNILIQMDLPLVPGDKIHCLDILFAFTKNVLGESGELDSLKTNMEEKFMATNLSKASYEPIATTLRWKQEDLSATVIQKAYRSYMLHRSLTLSNTLHVPRAEEDGVSLPGEGYITFMANSGLPDKSETASATSFPPSYDSVTRGLSDRANINPSSSMQNEDEVAAKEGNSPGPQ

SEQ ID NO: 3 - DNA, 5874 bp, Homo sapiens
  CDS (1)..(5874)
atggaattccccattggatccctcgaaactaacaacttccgtcgctttactccggagtcactggtggagatagagaagcaaattgctgccaagcagggaacaaagaaagccagagagaagcatagggagcagaaggaccaagaagagaagcctcggccccagctggacttgaaagcctgcaaccagctgcccaagttctatggtgagctcccagcagaactgatcggggagcccctggaggatctagatccgttctacagcacacaccggacatttatggtgctgaacaaagggaggaccatttcccggtttagtgccactcgggccctgtggctattcagtcctttcaacctgatcagaagaacggccatcaaagtgtctgtccactcgtggttcagtttatttattacggtcactattttggttaattgtgtgtgcatgacccgaactgaccttccagagaaaattgaatatgtcttcactgtcatttacacctttgaagccttgataaagatactggcaagaggattttgtctaaatgagttcacgtacctgagagatccttggaactggctggattttagcgtcattaccctggcatatgttggcacagcaatagatctccgtgggatctcaggcctgcggacattcagagttcttagagcattaaaaacagtttctgtgatcccaggcctgaaggtcattgtgggggccctgattcactcagtgaagaaactggctgatgtgaccatcctcaccatcttctgcctaagtgtttttgccttggtggggctgcaactcttcaagggcaacctcaaaaataaatgtgtcaagaatgacatggctgtcaatgagacaaccaactactcatctcacagaaaaccagatatctacataaataagcgaggcacttctgaccccttactgtgtggcaatggatctgactcaggccactgccctgatggttatatctgccttaaaacttctgacaacccggattttaactacaccagctttgattcctttgcttgggctttcctctcactgttccgcctcatgacacaggattcctgggaacgcctctaccagcagaccctgaggacttctgggaaaatctatatgatcttttttgtgctcgtaatcttcctgggatctttctacctggtcaacttgatcttggctgtagtcaccatggcgtatgaggagcagaaccaggcaaccactgatgaaattgaagcaaaggagaagaagttccaggaggccctcgagatgctccggaaggagcaggaggtgctagcagcactagggattgacacaacctctctccactcccacaatggatcacctttaacctccaaaaatgccagtgagagaaggcatagaataaagccaagagtgtcagagggctccacagaagacaacaaatcaccccgctctgatccttacaaccagcgcaggatgtcttttctaggcctcgcctctggaaaacgccgggctagtcatggcagtgtgttccatttccggtcccctggccgagatatctcactccctgagggagtcacagatgatggagtctttcctggagaccacgaaagccatcggggctctctgctgctgggtgggggtgctggccagcaaggccccctccctagaagccctcttcctcaacccagcaaccctgactccaggcatggagaagatgaacaccaaccgccgcccactagtgagcttgcccctggagctgtcgatgtctcggcattcgatgcaggacaaaagaagactttcttgtcagcagaatacttagatgaacctttccgggcccaaagggcaatgagtgttgtcagtatcataacctccgtccttgaggaactcgaggagtctgaacagaagtgcccaccctgcttgaccagcttgtctcagaagtatctgatctgggattgctgccccatgtgggtgaagctcaagacaattctctttgggcttgtgacggatccctttgcagagctcaccatcaccttgtgcatcgtggtgaacaccatcttcatggccatggagcaccatggcatgagccctaccttcgaagccatgctccagataggcaacatcgtctttaccatattttttactgctgaaatggtcttcaaaatcattgccttcgacccatactattatttccagaagaagtggaatatctttgactgcatcatcgtcactgtgagtctgctagagctgggcgtggccaagaagggaagcctgtctgtgctgcggagcttccgcttgctgcgcgtattcaagctggccaaatcctggcccaccttaaacacactcatcaagatcatcggaaactcagtgggggcactggggaacctcaccatcatcctggccatcattgtctttgtctttgctctggttggcaagcagctcctaggggaaaactaccgtaacaaccgaaaaaatatctccgcgccccatgaagactggccccgctggcacatgcacgacttcttccactctttcctcattgtcttccgtatcctctgtggagagtggattgagaacatgtgggcctgcatggaagttggccaaaaatccatatgcctcatccttttcttgacggtgatggtgctagggaacctggtggtgcttaacctgttcatcgccctgctattgaactctttcagtgctgacaacctcacagccccggaggacgatggggaggtgaacaacctgcaggtggccctggcacggatccaggtctttggccatcgtaccaaacaggctctttgcagcttcttcagcaggtcctgcccattcccccagcccaaggcagagcctgagctggtggtgaaactcccactctccagctccaaggctgagaaccacattgctgccaacactgccagggggagctctggagggctccaagctcccagaggccccagggatgagcacagtgacttcatcgctaatccgactgtgtgggtctctgtgcccattgctgagggtgaatctgatcttgatgacttggaggatgatggtggggaagatgctcagagcttccagcaggaagtgatccccaaaggacagcaggagcagctgcagcaagtcgagaggtgtggggaccacctgacacccaggagcccaggcactggaacatcttctgaggacctggctccatccctgggtgagacgtggaaagatgagtctgttcctcaggcccctgctgagggagtggacgacacaagctcctctgagggcagcacggtggactgcctagatcctgaggaaatcctgaggaagatccctgagctggcagatgacctggaagaaccagatgactgcttcacagaaggatgcattcgccactgtccctgctgcaaactggataccaccaagagtccatgggatgtgggctggcaggtgcgcaagacttgctaccgtatcgtggagcacagctggtttgagagcttcatcatcttcatgatcctgctcagcagtggatctctggcctttgaagactattacctggaccagaagcccacggtgaaagctttgctggagtacactgacagggtcttcacctttatctttgtgttcgagatgctgcttaagtgggtggcctatggcttcaaaaagtacttcaccaatgcctggtgctggctggacttcctcattgtgaatatctcactgataagtctcacagcgaagattctggaatattctgaagtggctcccatcaaagcccttcgaacccttcgcgctctgcggccactgcgggctctttctcgatttgaaggcatgcgggtggtggtggatgccctggtgggcgccatcccatccatcatgaatgtcctcctcgtctgcctcatcttctggctcatcttcagcatcatgggtgtgaacctcttcgcagggaagttttggaggtgcatcaactataccgatggagagttttcccttgtacctttgtcgattgtgaataacaagtctgactgcaagattcaaaactccactggcagcttcttctgggtcaatgtgaaagtcaactttgataatgttgcaatgggttaccttgcacttctgcaggtggcaacctttaaaggctggatggacattatgtatgcagctgttgattcccgggaggtcaacatgcaacccaagtgggaggacaacgtgtacatgtatttgtactttgtcatcttcatcatttttggaggcttcttcacactgaatctctttgttggggtcataattgacaacttcaatcaacagaaaaaaaagttagggggccaggacatcttcatgacagaggagcagaagaaatactacaatgccatgaagaagttgggctccaagaagccccagaagcccatcccacggcccctgaacaagttccagggttttgtctttgacatcgtgaccagacaagcttttgacatcaccatcatggtcctcatctgcctcaacatgatcaccatgatggtggagactgatgaccaaagtgaagaaaagacgaaaattctgggcaaaatcaaccagttctttgtggccgtcttcacaggcgaatgtgtcatgaagatgttcgctttgaggcagtactacttcacaaatggctggaatgtgtttgacttcattgtggtggttctctccattgcgagcctgattttttctgcaattcttaagtcacttcaaagttacttctccccaacgctcttcagagtcatccgcctggcccgaattggccgcatcctcagactgatccgagcggccaaggggatccgcacactgctctttgccctcatgatgtccctgcctgccctcttcaacatcgggctgttgctattccttgtcatgttcatctactccatcttcggtatgtccagctttccccatgtgaggtgggaggctggcatcgacgacatgttcaacttccagaccttcgccaacagcatgctgtgcctcttccagattaccacgtcggccggctgggatggcctcctcagccccatcctcaacacagggcccccctactgtgaccccaatctgcccaacagcaatggcaccagaggggactgtgggagcccagccgtaggcatcatcttcttcaccacctacatcatcatctccttcctcatcgtggtcaacatgtacattgcagtgattctggagaacttcaatgtggccacggaggagagcactgagcctctgagtgaggacgactttgacatgttctatgagacctgggagaagtttgacccagaggccactcagtttattaccttttctgctctctcggactttgcagacactctctctggtcccctgagaatcccaaaacccaatcgaaatatactgatccagatggacctgcctttggtccctggagataagatccactgcttggacatcctttttgctttcaccaagaatgtcctaggagaatccggggagttggattctctgaaggcaaatatggaggagaagtttatggcaactaatctttcaaaatcatcctatgaaccaatagcaaccactctccgatggaagcaagaagacatttcagccactgtcattcaaaaggcctatcggagctatgtgctgcaccgctccatggcactctctaacaccccatgtgtgcccagagctgaggaggaggctgcatcactcccagatgaaggttttgttgcattcacagcaaatgaaaattgtgtactcccagacaaatctgaaactgcttctgccacatcattcccaccgtcctatgagagtgtcactagaggccttagtgatagagtcaacatgaggacatctagctcaatacaaaatgaagatgaagccaccagtatggagctgattgcccctgggccctagtga

SEQ ID NO: 4 - protein, 1956 aa, Homo sapiens
MEFPIGSLETNNFRRFTPESLVEIEKQIAAKQGTKKAREKHREQKDQEEKPRPQLDLKACNQLPKFYGELPAELIGEPLEDLDPFYSTHRTFMVLNKGRTISRFSATRALWLFSPFNLIRRTAIKVSVHSWFSLFITVTILVNCVCMTRTDLPEKIEYVFTVIYTFEALIKILARGFCLNEFTYLRDPWNWLDFSVITLAYVGTAIDLRGISGLRTFRVLRALKTVSVIPGLKVIVGALIHSVKKLADVTILTIFCLSVFALVGLQLFKGNLKNKCVKNDMAVNETTNYSSHRKPDIYINKRGTSDPLLCGNGSDSGHCPDGYICLKTSDNPDFNYTSFDSFAWAFLSLFRLMTQDSWERLYQQTLRTSGKIYMIFFVLVIFLGSFYLVNLILAVVTMAYEEQNQATTDEIEAKEKKFQEALEMLRKEQEVLAALGIDTTSLHSHNGSPLTSKNASERRHRIKPRVSEGSTEDNKSPRSDPYNQRRMSFLGLASGKRRASHGSVFHFRSPGRDISLPEGVTDDGVFPGDHESHRGSLLLGGGAGQQGPLPRSPLPQPSNPDSRHGEDEHQPPPTSELAPGAVDVSAFDAGQKKTFLSAEYLDEPFRAQRAMSVVSIITSVLEELEESEQKCPPCLTSLSQKYLIWDCCPMWVKLKTILFGLVTDPFAELTITLCIVVNTIFMAMEHHGMSPTFEAMLQIGNIVFTIFFTAEMVFKIIAFDPYYYFQKKWNIFDCIIVTVSLLELGVAKKGSLSVLRSFRLLRVFKLAKSWPTLNTLIKIIGNSVGALGNLTIILAIIVFVFALVGKQLLGENYRNNRKNISAPHEDWPRWHMHDFFHSFLIVFRILCGEWIENMWACMEVGQKSICLILFLTVMVLGNLVVLNLFIALLLNSFSADNLTAPEDDGEVNNLQVALARIQVFGHRTKQALCSFFSRSCPFPQPKAEPELVVKLPLSSSKAENHIAANTARGSSGGLQAPRGPRDEHSDFIANPTVWVSVPIAEGESDLDDLEDDGGEDAQSFQQEVIPKGQQEQLQQVERCGDHLTPRSPGTGTSSEDLAPSLGETWKDESVPQAPAEGVDDTSSSEGSTVDCLDPEEILRKIPELADDLEEPDDCFTEGCIRHCPCCKLDTTKSPWDVGWQVRKTCYRIVEHSWFESFIIFMILLSSGSLAFEDYYLDQKPTVKALLEYTDRVFTFIFVFEMLLKWVAYGFKKYFTNAWCWLDFLIVNISLISLTAKILEYSEVAPIKALRTLRALRPLRALSRFEGMRVVVDALVGAIPSIMNVLLVCLIFWLIFSIMGVNLFAGKFWRCINYTDGEFSLVPLSIVNNKSDCKIQNSTGSFFWVNVKVNFDNVAMGYLALLQVATFKGWMDIMYAAVDSREVNMQPKWEDNVYMYLYFVIFIIFGGFFTLNLFVGVIIDNFNQQKKKLGGQDIFMTEEQKKYYNAMKKLGSKKPQKPIPRPLNKFQGFVFDIVTRQAFDITIMVLICLNMITMMVETDDQSEEKTKILGKINQFFVAVFTGECVMKMFALRQYYFTNGWNVFDFIVVVLSIASLIFSAILKSLQSYFSPTLFRVIRLARIGRILRLIRAAKGIRTLLFALMMSLPALFNIGLLLFLVMFIYSIFGMSSFPHVRWEAGIDDMFNFQTFANSMLCLFQITTSAGWDGLLSPILNTGPPYCDPNLPNSNGTRGDCGSPAVGIIFFTTYIIISFLIVVNMYIAVILENFNVATEESTEPLSEDDFDMFYETWEKFDPEATQFITFSALSDFADTLSGPLRIPKPNRNILIQMDLPLVPGDKIHCLDILFAFTKNVLGESGELDSLKANMEEKFMATNLSKSSYEPIATTLRWKQEDISATVIQKAYRSYVLHRSMALSNTPCVPRAEEEAASLPDEGFVAFTANENCVLPDKSETASATSFPPSYESVTRGLSDRVNMRTSSSIQNEDEATSMELIAPGP

SEQ ID NO: 5 - DNA, 8301 bp, Rattus norvegicus
  CDS (1)..(8301)
atgcccatcacccaggacaatgccttgctgcacctgcccctcctgtacgagtggctgcagaacagcctgagggagggtggggacagtccggagcagcggctctgccaggcggccatccagaagctgcaggagtacatccaactgaacttggctgtggatgagagtacagttccccctgatcacagtcccccggagatggagatctgtacggtgtatctcaccaagcagctgggggacactgagactgtggggctcagttttggcaacatccctgttttcggggactacggtgaaaagcgtcgagggggtaagaagaggaaaacccaccaggggccagtgctggacgtgggctgcatctgggtgacagagctgaggaagaacagcccagcggggaagagcggaaaagttcggctccgggatgagattctttccctgaatggacagctgatggtcggagttgatgtcactggggccagttacctcgctgagcagtgctggaatggcggcttcatctacctgatcatgctgcggcgcttcaagcagaaagcccacgtgacttacaatggcaacagtggcaacagctcagaacccggagagacaccgaccttggagctgggtgaccagacttcaaaaaagggaaaaagaacaagaaagtttggggtcatttccagaccctctatcagcaagacccctgaagactccaagagcagcagtggctgtgacacagccgatgatcccaattcggagttggagaacggcgcagaccccgaacttggaaatggccatgcctttgagctagaaaatggcccgcattctctcaaggatgtggctggaccccatctggagaggtcagaagcggacagtgaggtagagctcagagttccaaagacagaagcccctctgagtgacagcaatgacaaacgccgcttctcaaaaactgggaagacagacttccagtccagtgactgtctggcgcgggaggaagttggccggatatggaagatggagctgctcaaagaatcagatgggctgggaattcaggttagtggaggccgaggatcaaagcgctcacctcacgctatcgttgtcacccaagtgaaggaaggaggtgccgctcacagggatggcaggctgtccttaggagacgaactgctggtgatcaatggtcacttactggtcgggctgtcccacgaggaagctgtggccattctgcgctcagccactgggatggtacagctggtagtggccagcaagatgcccgggtcagaagaatcccaggacgtaggcagctctgaggaatccaaagggaacttggaaagtcccaaacagggcaactgtaagacgaaactcaagagccgactctcaggaggtgtccaccgcctggagtctgttgaagaatataatgaactgatggtacgaaatggggacccccggatcaggatgctggaggtctcccgagatggccggaagcactctcttccgcagctgctggactctactgggacatctcaggaataccacatcgtgaagaagtctacccgctccctgagcaccacccacgtggaatcaccgtggaggctcatccgaccttctgtcatttccatcatcgggctatacaaagagaaaggcaagggccttggctttagcattgctggagggcgagactgcattcgaggccagatgggaatttttgtcaagaccattttcccaaacggatcagccgcagaggacggcaggctcaaagaaggagatgaaatcctagatgtaaatggaataccaatcaagggcttgacgtttcaagaggctattcacaccttcaagcaaatccgaagtgggctgtttgtcctcaccgtgcgcaccaagctcctcagccccagtctcacgccctgctccactcccacgcacatgagcagatcgagctccccaagcttcaacaccaacagtgggggaaccccagcaggaggaggccaagaggaaggtggctcttcatccctgggtcggaaggctcccgggcccaaagacaggattgtcatggaagtcacactcaacaaagagccaagagttggactgggcattggtgcctgctgcttggccttggaaaacagccctccaggtatatacattcacagccttgcccctggatccgtggccaagatggagagcaacctgagccggggagaccaaatcctggaagtgaattctgtgaacgtgcgtcatgcggctttaagcaaagtgcatgccatcttaagtaaatgtcccccagggcctgttcgcctggtcatcggccgacaccctaatccaaaggtctcggagcaggaaatggacgaagtgatagcacgcagcacttaccaggaaagcagagaagccaactcctcccccggcctcggtacccccttgaagagcccctctctggccaaaaaggactcactcctctctgaatctgagctctcccaatactttgtccatgatggccagggctccctgtcagactttgtggtggctggctctgaggatgaggatcaccctggaagtggatatgagacctcggaggatggcagcctgcttcctgtcccctcagctcacaaagccagggccaacagccttgtgacccttggaagccagaggacttctgggctcttacacaagcaggtgacagttgccaggcaagccagtctccccggaagcccccaggtcctcaggaaccctcttctccgccagaggagggtgcgttgctatgacagcaatggtggcagcgatgatgaagacttcgatggtgaaggggactgcatctccctcccgggagtcctcccaggtcccggcaagcctctggtagaagatgacacgaggcctgccttgacaacctcttccaaaagtattgatgtgaacaagcaggaggaaagactccagaaaccactggtcagcaaggcctgctccgtgcctctcctcggcagctcgctggactcagagcacagcatcctcaatggagcagggggtacccctcccaaggtagccagcctgccaggttctggagaaacccccaagaatgggcccagaggctctgggagaaaagaaatgtcagggtcaagaagctcaccaaagctggaatacagagtccctacagacacccagagcccaaggagcccggaaaaccatacctccccaccacagaagagtgaaaatctggtgtccaggcacaagcccgtggccaggatcagcccacactacaagaggtctgatgcagaggaggccccaggtggaacagccaatggaccgtgtgctcaagacttgaaagtccaggcgtctcccgtgaaagatcctgtcaccagccgtcagccaggtggaaccgcagagaaggaacttcggggaaatcccaccccgggggacagctctgtccccaccaactgtggaccagccagtaccccatgccacccaaacattggactccccacagagaacccgcagggagctgcaccagagtgcgggccacaccctgggactggatgggacgggtcctcagagcatctctgttccccagggaagagcagggaggttcatcctgactccagcgagactcctacagtcgccgagcaagtccaccagcctgagagcctcagccagccagtgtctcccagaacctcagagcctgagtcccagggcatatctaaaatgaagccacccagtcagagatgtgtgtctcccagggagaaggcctcaactcctcccgactccagcagggcttgggccgctcctggggacagctctcctagcactaggagaatagctgtccccatgagcacaggagcagcaccagctactgccatcccacaggcctcccttgtgtcccaggaaaggagcagaggcctctcaggtcccagcaagggcctgggaactaaagaactctgcatacctaaaagcttgaaggacggtgctctgctcgaagacacagctcctgcatctggaaagatgtcacatgccagcagcccctctgggccagtggctactgaaaggaccctgtcaggaagcccagagaaccctgtgacagacatcgacaacttcattgaggaggcctctgaggccaggctttctcagtctcctcagaaagcagactgcagggctcacggggacacttttgaaagtcagccaccaggtggagctgggagcagcagttcccaccatgctcagatggtcaggagtgaccagacatcctccccaaggaagactggaggcacaggctcgcccccaccccagcagtgggccctccagccttccgtcctggattccatccatcctgacaaacatttggctgtgaacaaaaccttcttgaacaactactctagaaattttagcaatttccacgaagacagtatttccctttcaggcccgggcggcagttcagagccgtcgccctcatccatgtatggtaacgctgaagactcatcgtcagaccctgagtctcttgctgaagacccaggagcagctgccaggaacaactggtcacctcctctgtctcctgagtcctcccctaaagaaggtagcagtgagtctgaggacgagcgaatagaaatctgttccacagatggctgccctgggaccccagtgactgcccctcctcctacccaggttgcactctgcccagttctgccagtacagcagagggctgtgtgcaagccagtgggggacatctgtgagagagcctgcttcgtgccaggagcctcacgcacttccatcccggactcttctcagccattttccttcctggatgtaagctctgaggagccggagacatgggccagcataaatgcttcacagaaccacatgcccgtgtgcacagaaggaatcatggacgtcactagcacaagctcaaacatgggagacagccagtcttcccaaatgaccagacattgtcgaaacgcaccattcgtgcttgggaacccagatatggtaaatgatttgggacgtgacctgctggatgagggagccccaaaggaaggagcagctgctgcgagtgtgatgcgcagtgtgtttgccctgggggccgagggccctaagaacggagaggcggtcttggcagatctacacattgccgagcgcggcaacctggaagacttgctacagaaaccaaaaacaatctccaggaggcctatcctgacctggtttaaagaaataaataaagacagccaaggctcacatttgcggagcacatctgagaaagaacagtcctcgatgctggctctgggtcctggctcgaaagctaacatggtgaacaccggccacagaaagggggtgactgtgcctaagagtcctccttcacggcagaagagtcaggaaaataaagacctgccccccaaaagtccagtggaaacactaggtaactgtcagaaacccaagtgcagccctaaactgaagagactaaacagcaaaggcaaagccagtcctgaggtacccgtggccatttccactaagggcagcaggaacgaccacaggaagaccttgccttcaccccaggcctcccataaaatgttttctaaggcagtgtcacacaggctccacatagctgaccaggaagaacctaagaacactgctggggacacccccaagcctccccagtgcgtgccggagagcaagccaccccaggctgccttagggtcgctgaggacctccgcatccgacacaagcattagaacctttacttcgcccctgacctcccccaagcttctccctgagcagggtgcaaacagtaggttccacatggctgtctacttggaatctgacacaagctgtccaaccacctccaggtcccctaggagtggaccggagggcaaggcgccccatgctaactctgggtcagcgagtcccccagcatcgagggccagcctagccttggcagggatcaggcagagcaagcagttcacaccgggccgagcagacttgttagtatcagaagcaacccagccccagggcatctgtgagaaaggagctgaaaaaaaggttagtgatcctccacaaaggacaaaccagctaaaaatagttgagatttcttctgaaagagtgccaaagaatgcatgtggtgacaggccacccgaaagtgacagaaagggagggttcttgacccagaacaactgtcaggagaagagtgcaatcagactccggcagtcggaggaatcgtccccagagcatacacccttccctccctctcaggcctcccaagtggaacgggaaattcgatggtctttcagcatggccaaaccggccacctcttcctcctcctccctgcagctgcctgccaaattgccagagtccttccagggcaaatcaagccaaatgccagcctccgttggggtgcccaagaatggggtacccataggcctggccggagaagagagcccctacttcacaccgaggccagccaccaggacctattccatgccagcccagttctcgagccactttggacgagaaggtccttccccacacagcccaagtcactcgcctcaggacccgcaggtccctgcgatgggtggtaagctctctgagaagacagccaagggcgtgactaatggacagggcgtatatagtgtgaagcctctgctggaaacatcgaagaacctgtcaccagtggacggacgggatgtcagtgcagaccccgagacgagctgcctcatcccagacaaggtcaaagtcaccaggagacagtactgctgtgagcagagttggccccatgaatctacctcgtttttctctgtgaagcagagaatcaagtcttttgagaacttggccaattctgaccggcccacggccaagtgtgccacatccccgttcttatccgtaagctctaagcctcccattaacagacggtcgtctggcagcatcccttcagggagccccagcgatatgacctcgaggtcgctgaggcgcagcttgagttcctgcagtgaaagccagagtgaggccagcagccttctcccgcagatgacgaagtccccctccagcatgacgctgactgtctccaggcagaatccaccagacactagtaacaagggccccagtccagacccaaagaaatcacttgtccctgtgggaattcccacctccacagtgagcccagcctcacccagcaaaaggaacaagtcctctgtgcgccatgcccagccctctccagtatcccgatccaagctccaggagcggagaaccttaagcatgccagacctggacaagctgtgtaatggtgaggacgactctgccagcccaggggctgtgctctttaaaacccagctggagatcacccccagaaggtcgaaaggctcccaggctacttctccagctggctccccagctagaggccatgcagatttcaatggttcaaccttcctttcatgtcctatgaatggtgggaccagagcctacacaaaaggaaacagccctccagccagtgagcctgccatagccactggatccagggaagagggtgaatctgtgtgggccacgccttctgggaaaagctggtcggtgagtttggatcgactccttgcctcagtggggaaccagcagagattgcagggcattttatcattagtgggttctaaatcccccatcctcacgctcattcaggaagcgaaggcgcaatcagagactaaagaagatatctgcttcatagtcttgaataaaaaagaaggctcgggtctgggattcagtgtggcaggaggggccgacgtggagccaaaatcagtcatggtccacagggtgttttctcagggcgtggcttctcaggaagggactgtgagccgaggggacttccttctctccgtcaatggcacctccttagctggcttagcccacagtgaggtcacgaaggttctgcaccaggcagagctgcacaaacacgccctcatgattatcaagaaagggaatgaccaacccgggccctccttcaagcaggagcctccctcagccaatgggaaaggccctttccccagaaggaccttgccgctggagcctggagctgggagaaatggagctgctcacgatgcgctgtgtgttgaagtgctgaaaacctctgctgggctgggattgagtctggatggaggaaaatcgtctgtatccggagaggggccactggtgattaaaagggtgtacaaaggcggtgcggccgaacgagctggaacaatagaagcgggtgatgagatcctagccattaatgggaagcccttggtcgggctggtgcacttcgatgcctggaacatcatgaagtctgttccagaagggcccgtgcagctagtgatcagaaagcacagggattcgtga

SEQ ID NO: 6 - protein, 2766 aa, Rattus norvegicus
MPITQDNALLHLPLLYEWLQNSLREGGDSPEQRLCQAAIQKLQEYIQLNLAVDESTVPPDHSPPEMEICTVYLTKQLGDTETVGLSFGNIPVFGDYGEKRRGGKKRKTHQGPVLDVGCIWVTELRKNSPAGKSGKVRLRDEILSLNGQLMVGVDVTGASYLAEQCWNGGFIYLIMLRRFKQKAHVTYNGNSGNSSEPGETPTLELGDQTSKKGKRTRKFGVISRPSISKTPEDSKSSSGCDTADDPNSELENGADPELGNGHAFELENGPHSLKDVAGPHLERSEADSEVELRVPKTEAPLSDSNDKRRFSKTGKTDFQSSDCLAREEVGRIWKMELLKESDGLGIQVSGGRGSKRSPHAIVVTQVKEGGAAHRDGRLSLGDELLVINGHLLVGLSHEEAVAILRSATGMVQLVVASKMPGSEESQDVGSSEESKGNLESPKQGNCKTKLKSRLSGGVHRLESVEEYNELMVRNGDPRIRMLEVSRDGRKHSLPQLLDSTGTSQEYHIVKKSTRSLSTTHVESPWRLIRPSVISIIGLYKEKGKGLGFSIAGGRDCIRGQMGIFVKTIFPNGSAAEDGRLKEGDEILDVNGIPIKGLTFQEAIHTFKQIRSGLFVLTVRTKLLSPSLTPCSTPTHMSRSSSPSFNTNSGGTPAGGGQEEGGSSSLGRKAPGPKDRIVMEVTLNKEPRVGLGIGACCLALENSPPGIYIHSLAPGSVAKMESNLSRGDQILEVNSVNVRHAALSKVHAILSKCPPGPVRLVIGRHPNPKVSEQEMDEVIARSTYQESREANSSPGLGTPLKSPSLAKKDSLLSESELSQYFVHDGQGSLSDFVVAGSEDEDHPGSGYETSEDGSLLPVPSAHKARANSLVTLGSQRTSGLLHKQVTVARQASLPGSPQVLRNPLLRQRRVRCYDSNGGSDDEDFDGEGDCISLPGVLPGPGKPLVEDDTRPALTTSSKSIDVNKQEERLQKPLVSKACSVPLLGSSLDSEHSILNGAGGTPPKVASLPGSGETPKNGPRGSGRKEMSGSRSSPKLEYRVPTDTQSPRSPENHTSPPQKSENLVSRHKPVARISPHYKRSDAEEAPGGTANGPCAQDLKVQASPVKDPVTSRQPGGTAEKELRGNPTPGDSSVPTNCGPASTPCHPNIGLPTENPQGAAPECGPHPGTGWDGSSEHLCSPGKSREVHPDSSETPTVAEQVHQPESLSQPVSPRTSEPESQGISKMKPPSQRCVSPREKASTPPDSSRAWAAPGDSSPSTRRIAVPMSTGAAPATAIPQASLVSQERSRGLSGPSKGLGTKELCIPKSLKDGALLEDTAPASGKMSHASSPSGPVATERTLSGSPENPVTDIDNFIEEASEARLSQSPQKADCRAHGDTFESQPPGGAGSSSSHHAQMVRSDQTSSPRKTGGTGSPPPQQWALQPSVLDSIHPDKHLAVNKTFLNNYSRNFSNFHEDSISLSGPGGSSEPSPSSMYGNAEDSSSDPESLAEDPGAAARNNWSPPLSPESSPKEGSSESEDERIEICSTDGCPGTPVTAPPPTQVALCPVLPVQQRAVCKPVGDICERACFVPGASRTSIPDSSQPFSFLDVSSEEPETWASINASQNHMPVCTEGIMDVTSTSSNMGDSQSSQMTRHCRNAPFVLGNPDMVNDLGRDLLDEGAPKEGAAAASVMRSVFALGAEGPKNGEAVLADLHIAERGNLEDLLQKPKTISRRPILTWFKEINKDSQGSHLRSTSEKEQSSMLALGPGSKANMVNTGHRKGVTVPKSPPSRQKSQENKDLPPKSPVETLGNCQKPKCSPKLKRLNSKGKASPEVPVAISTKGSRNDHRKTLPSPQASHKMFSKAVSHRLHIADQEEPKNTAGDTPKPPQCVPESKPPQAALGSLRTSASDTSIRTFTSPLTSPKLLPEQGANSRFHMAVYLESDTSCPTTSRSPRSGPEGKAPHANSGSASPPASRASLALAGIRQSKQFTPGRADLLVSEATQPQGICEKGAEKKVSDPPQRTNQLKIVEISSERVPKNACGDRPPESDRKGGFLTQNNCQEKSAIRLRQSEESSPEHTPFPPSQASQVEREIRWSFSMAKPATSSSSSLQLPAKLPESFQGKSSQMPASVGVPKNGVPIGLAGEESPYFTPRPATRTYSMPAQFSSHFGREGPSPHSPSHSPQDPQVPAMGGKLSEKTAKGVTNGQGVYSVKPLLETSKNLSPVDGRDVSADPETSCLIPDKVKVTRRQYCCEQSWPHESTSFFSVKQRIKSFENLANSDRPTAKCATSPFLSVSSKPPINRRSSGSIPSGSPSDMTSRSLRRSLSSCSESQSEASSLLPQMTKSPSSMTLTVSRQNPPDTSNKGPSPDPKKSLVPVGIPTSTVSPASPSKRNKSSVRHAQPSPVSRSKLQERRTLSMPDLDKLCNGEDDSASPGAVLFKTQLEITPRRSKGSQATSPAGSPARGHADFNGSTFLSCPMNGGTRAYTKGNSPPASEPAIATGSREEGESVWATPSGKSWSVSLDRLLASVGNQQRLQGILSLVGSKSPILTLIQEAKAQSETKEDICFIVLNKKEGSGLGFSVAGGADVEPKSVMVHRVFSQGVASQEGTVSRGDFLLSVNGTSLAGLAHSEVTKVLHQAELHKHALMIIKKGNDQPGPSFKQEPPSANGKGPFPRRTLPLEPGAGRNGAAHDALCVEVLKTSAGLGLSLDGGKSSVSGEGPLVIKRVYKGGAAERAGTIEAGDEILAINGKPLVGLVHFDAWNIMKSVPEGPVQLVIRKHRDS

SEQ ID NO: 7 - DNA, 4641 bp, Rattus norvegicus
  CDS (273)..(4424)
ggaactctggaggtgtctggaggcccactgagccccagacccaggaggcccaagtagctggaactgaccctcaggcagcaagacctcaaaggaagagtgaaattccggcttacagctgtacatcaccccgagtggggctcctgtcaggagaaaagaccatcacccaggaagcggctcagcggcagctgctccatgaggagctgaagctggtcctacagcagaaggaggagaggaaacaggagcctgagccccaggtgactctctgcagagctatggaggccaggagccgcagcgctgaggagctgagacgagcggagttggtggagattatcgtggagacagaggcgcagaccggggtcagcggcttcaatgtagcaggcggcggcaaagaaggaatctttgtccgcgagctgcgagaggactcaccggccgccaagagcctcagtttgcaggaaggggaccaacttctgagcgcccgtgtgttctttgagaacttcaaatatgaggatgcactacgcctgctgcaatgtgccgagccctacaaggtctccttctgcttgaagcgcactgtgcccaccggggacctggcactgcggcccgggacggtgtctggatacgagatgaagggcccgcgggccaaggtggccaagctgaacatccagagtctgtcccctgtgaagaagaagaagatggtgattgggaccctggggacccctgcagatttggcccctgttgacgtcgagttctcttttcccaagttctcccgattgcgtcggggccttaaagccgatgctgtcaagggacctgtcccagctgcccctgcccgacgacgtctccagctgcctcggctacgtgtccgagaagtagctgaagaggcccaggtagcccgaatggctgctgctgctcctccctctaggaaggccaagtcagaggctgaggtagccacaggggctggattcacagcccctcagatagagctagttgggcctcggctgcctagcgcagaggtgggtgtccctaaggtctcagttcccaagggaaccccatcaacagaggcagccagcggctttgcccttcacctgccaacccttgggctaggagccccagctgcaccggctgtggagcccccaaccacaggaatccaggtcccgcaagtggaactccccaccctgccctctttacccactctgcccacacttccgtgcctagatacccaggaaggggctgcagtggtcaaagtccccaccctggatgtggcagctccgtctgtggaggtggacctggctttgccaggtgcagaggtggaggcccagggagaggtacctgaagtggctctcaagatgccccgtctcagtttcccccgttttggggttcgagggaaggaagctactgaagccaaggtagtcaagggcagccctgaggccaaagcaaagggtcccagacttcgaatgcccacctttgggctttctctcctggaatcccggccctctggccctgaagttgctgctgagagcaagctgaagctacccaccctcaagatgccctctttcggcatcagcgtagctgggcctgaggtcaaggcacccaaagggcctgaagtgaagctccccaaagttcctgagatcaaactcccgaaagcgccagaggcagccattccagatgtgcaactccccgaggtacagctgcccaaaatgtcagacatgaaacttccaaagatccctgagatggctgtacccgatgttcaccttccggaagtgaagctgcccaaagtccccgagatgaaagtcccagaaatgaagcttccgaagatcccggagatggccgtgcctgatgtacaccttccagatatacagctcccgaaagttcccgagatgaagctcccagacatgaagctcccgaaggtgcctgagatggccgtgcctgatgtacaccttccagatatacagctcccgaaagttcccgagatgaagctcccagacatgaagctcccgaaggtgcctgagatggccgtgcctgatgtacgaattccggaagttcagctacccaaagtgtccgaggtgaagctcccgaagataccggacatggccgtgcctgatgttcgcctcccagagctgcaactgcccaaaatgtctgaggtgaagctcccgaagataccggacatggccgtacctgatgttcgcctcccagaagttcagctacccaaagtgtcagagctgaagctcccgaaggtgcctgagatgaccatgcccgacattcgcctcccggaagttcagctgcccaaagtgcctgacattaaacttccagaaataaaactccccaaagtgcctgagatggccgtgcctgatgtcccccttccagaactacagctgcccaaagtgccacaggtcccagacgtgcatcttcccaaagtgccagagatgaagttgcccaaggttcctgaggcacagaggaaatctgcaggggcggagcaggcagaaaagaccgaatttagcttcaagttgcccaagatgactgtgcccaagttggggaaagtgaccaagcctggggaggcaggtattgaggttccagacaaactcctgatacttccctgtctgcagccagaggtgggcactgaggtggcccgtgttggtgtcccttccctctctctcccttctgtggagcttgacttgcctggggccctgggcctggagggacaagtccaagaagctgtctctggcaaagtggagaagccagagggccccagggtggcagtagggactggagaggcgggcttccgcgtgccctctgtggagattgtcaatcctcagctgcccacggttgaagtcaagaaagagcagctagagatggtggagatgaaagtcaaacccacttccaagttctctctgcccaaatttggactttcagggcccaaagctgtcaaggcagaggtggaggggcctgggcgagccaccaagctgaaggtatccaagtttgccatctcgcttcccagagctcgagcagggactgacgcggacgcgaagggagctggggaagcggggttgctgcctgccctcgatctgtccatcccacagctcagcctggatgctcaactgccctcaggcaaggtggaggtagcaggggctgagagcaagcctaaagggtccagatttgctctgcccaagtttggggcgaaaggccgggactctgaagccgacgtactggtggcaggggaggctgagctggaggggaagggttggggctgggacgggaaggtgaagatgcccaagctgaagatgccatcttttgggctgtcccgaggaaaagaagcagaaattcaggatgggcgtgtcagcccaggagaaaagctggaagccatagctgggcagcttaagatccctgaggtggaactggtcacaccaggagctcaggagacagagaaggtcaccagtggagtgaagccatcaggcctccaggtgtccaccactaggcaggtggttgcagagggccaggagggggcgcagagggtgtcctcattaggtatctctttgccccaggtggaactggccagctttggggaggcaggccctgagatcgcagccccatctgcagagggcacagtaggctctaggatccaggtgccacaggtgatgctggagttgccgggaacccaggtggcagggggtgatctgttagtgggtgagggcatcttcaagatgcccacagtgacagtgccccagttagagctggatgtggggttgggccatgaagcccaggctggtgaaacagccaagagtgagggcgggttaaagctgaagttgcccacactgggggcaggaggcaaaggagagggtgctgaggcccagagccccgaggcccagcacaccttccacatctcattgcctgacgtagaactcacatcaccagtgagtagccacgctgagtaccaggtggttgagggcgatggggatggcgggcacaaactcaaggtgcggctgcccctgtttggtctggcaagggccaaggaaggaatagaaactggagaaaaggttaaaagtccaaagctcaggctaccccgagtgggcttcagccaaagtgagtcggcctctggagaaggctctcccagtcctgaggaggaggaagaaggcagtggggaaggggcttccggtcgccgtggtcgggtcagggtccgcttgcctcgtgtaggcttggcttccccttctaaaggctctaagggacaggagggtgatgcggcctccaagtccccagttggggagaagtcccccaagttccgctttcctagggtgtccttaagccccaaggcccggagtgggagtaaggaccgggaagaaggtggattcagggtccgactgcccagtgtgggattttcagaaacagcagctccaggctccgccaggattgaggggacccaggctgctgccatctgaagccctgggacagctgtggattccccctcttgtcttcccatccccatccctgctccccattttatgtgtgacattactagcactaatcctcagagggcttgaaggtgggcagctgactcaggcaggagcggtctgtgccacctcattggctgacgtgcctgtatatcatgccaagctctgtgaataaaataattcaaaagttaaaaaaaaaaaaaaa

SEQ ID NO: 8 - protein, 1383 aa, Rattus norvegicus
MEARSRSAEELRRAELVEIIVETEAQTGVSGFNVAGGGKEGIFVRELREDSPAAKSLSLQEGDQLLSARVFFENFKYEDALRLLQCAEPYKVSFCLKRTVPTGDLALRPGTVSGYEMKGPRAKVAKLNIQSLSPVKKKKMVIGTLGTPADLAPVDVEFSFPKFSRLRRGLKADAVKGPVPAAPARRRLQLPRLRVREVAEEAQVARMAAAAPPSRKAKSEAEVATGAGFTAPQIELVGPRLPSAEVGVPKVSVPKGTPSTEAASGFALHLPTLGLGAPAAPAVEPPTTGIQVPQVELPTLPSLPTLPTLPCLDTQEGAAVVKVPTLDVAAPSVEVDLALPGAEVEAQGEVPEVALKMPRLSFPRFGVRGKEATEAKVVKGSPEAKAKGPRLRMPTFGLSLLESRPSGPEVAAESKLKLPTLKMPSFGISVAGPEVKAPKGPEVKLPKVPEIKLPKAPEAAIPDVQLPEVQLPKMSDMKLPKIPEMAVPDVHLPEVKLPKVPEMKVPEMKLPKIPEMAVPDVHLPDIQLPKVPEMKLPDMKLPKVPEMAVPDVHLPDIQLPKVPEMKLPDMKLPKVPEMAVPDVRIPEVQLPKVSEVKLPKIPDMAVPDVRLPELQLPKMSEVKLPKIPDMAVPDVRLPEVQLPKVSELKLPKVPEMTMPDIRLPEVQLPKVPDIKLPEIKLPKVPEMAVPDVPLPELQLPKVPQVPDVHLPKVPEMKLPKVPEAQRKSAGAEQAEKTEFSFKLPKMTVPKLGKVTKPGEAGIEVPDKLLILPCLQPEVGTEVARVGVPSLSLPSVELDLPGALGLEGQVQEAVSGKVEKPEGPRVAVGTGEAGFRVPSVEIVNPQLPTVEVKKEQLEMVEMKVKPTSKFSLPKFGLSGPKAVKAEVEGPGRATKLKVSKFAISLPRARAGTDADAKGAGEAGLLPALDLSIPQLSLDAQLPSGKVEVAGAESKPKGSRFALPKFGAKGRDSEADVLVAGEAELEGKGWGWDGKVKMPKLKMPSFGLSRGKEAEIQDGRVSPGEKLEAIAGQLKIPEVELVTPGAQETEKVTSGVKPSGLQVSTTRQVVAEGQEGAQRVSSLGISLPQVELASFGEAGPEIAAPSAEGTVGSRIQVPQVMLELPGTQVAGGDLLVGEGIFKMPTVTVPQLELDVGLGHEAQAGETAKSEGGLKLKLPTLGAGGKGEGAEAQSPEAQHTFHISLPDVELTSPVSSHAEYQVVEGDGDGGHKLKVRLPLFGLARAKEGIETGEKVKSPKLRLPRVGFSQSESASGEGSPSPEEEEEGSGEGASGRRGRVRVRLPRVGLASPSKGSKGQEGDAASKSPVGEKSPKFRFPRVSLSPKARSGSKDREEGGFRVRLPSVGFSETAAPGSARIEGTQAAAI

SEQ ID NO: 9 - DNA, 1901 bp, Homo sapiens
  CDS (34)..(1728)
gtctttccggcggtgctcgcaagcgaggcagccatgtcttatcccgctgatgattatgagtctgaggcggcttatgacccctacgcttatcccagcgactatgatatgcacacaggagatccaaagcaggaccttgcttatgaacgtcagtatgaacagcaaacctatcaggtgatccctgaggtgatcaaaaacttcatccagtatttccacaaaactgtctcagatttgattgaccagaaagtgtatgagctacaggccagtcgtgtctccagtgatgtcattgaccagaaggtgtatgagatccaggacatctatgagaacagctggaccaagctgactgaaagattcttcaagaatacaccttggcccgaggctgaagccattgctccacaggttggcaatgatgctgtcttcctgattttatacaaagaattatactacaggcacatatatgccaaagtcagtgggggaccttccttggagcagaggtttgaatcctattacaactactgcaatctcttcaactacattcttaatgccgatggtcctgctccccttgaactacccaaccagtggctctgggatattatcgatgagttcatctaccagtttcagtcattcagtcagtaccgctgtaagactgccaagaagtcagaggaggagattgactttcttcgttccaatcccaaaatctggaatgttcatagtgtcctcaatgtccttcattccctggtagacaaatccaacatcaaccgacagttggaggtatacacaagcggaggtgaccctgagagtgtggctggggagtatgggcggcactccctctacaaaatgcttggttacttcagcctggtcgggcttctccgcctgcactccctgttaggagattactaccaggccatcaaggtgctggagaacatcgaactgaacaagaagagtatgtattcccgtgtgccagagtgccaggtcaccacatactattatgttgggtttgcatatttgatgatgcgtcgttaccaggatgccatccgggtcttcgccaacatcctcctctacatccagaggaccaagagcatgttccagaggaccacgtacaagtatgagatgattaacaagcagaatgagcagatgcatgcgctgctggccattgccctcacgatgtaccccatgcgtatcgatgagagcattcacctccagctgcgggagaaatatggggacaagatgttgcgcatgcagaaaggtgacccacaagtctatgaagaacttttcagttactcctgccccaagttcctgtcgcctgtagtgcccaactatgataatgtgcaccccaactaccacaaagagcccttcctgcagcagctgaaggtgttttctgatgaagtacagcagcaggcccagctttcaaccatccgcagcttcctgaagctctacaccaccatgcctgtggccaagctggctggcttcctggacctcacagagcaggagttccggatccagcttcttgtcttcaaacacaagatgaagaacctcgtgtggaccagcggtatctcagccctggatggtgaatttcagtcagcctcagaggttgacttctacattgataaggacatgatccacatcgcggacaccaaggtcgccaggcgttatggggatttcttcatccgtcagatccacaaatttgaggagcttaatcgaaccctgaagaagatgggacagagaccttgatgatattcacacacattcaggaacctgttttgatgtattataggcaggaagtgtttttgctaccgtgaaacctttacctagatcagccatcagcctgtcaactcagttaacaagttaaggaccgaagtgtttcaagtggatctcagtaaaggatctttggagccagaaaaaaa

SEQ ID NO: 10 - protein, 564 aa, Homo sapiens
MSYPADDYESEAAYDPYAYPSDYDMHTGDPKQDLAYERQYEQQTYQVIPEVIKNFIQYFHKTVSDLIDQKVYELQASRVSSDVIDQKVYEIQDIYENSWTKLTERFFKNTPWPEAEAIAPQVGNDAVFLILYKELYYRHIYAKVSGGPSLEQRFESYYNYCNLFNYILNADGPAPLELPNQWLWDIIDEFIYQFQSFSQYRCKTAKKSEEEIDFLRSNPKIWNVHSVLNVLHSLVDKSNINRQLEVYTSGGDPESVAGEYGRHSLYKMLGYFSLVGLLRLHSLLGDYYQAIKVLENIELNKKSMYSRVPECQVTTYYYVGFAYLMMRRYQDAIRVFANILLYIQRTKSMFQRTTYKYEMINKQNEQMHALLAIALTMYPMRIDESIHLQLREKYGDKMLRMQKGDPQVYEELFSYSCPKFLSPVVPNYDNVHPNYHKEPFLQQLKVFSDEVQQQAQLSTIRSFLKLYTTMPVAKLAGFLDLTEQEFRIQLLVFKHKMKNLVWTSGISALDGEFQSASEVDFYIDKDMIHIADTKVARRYGDFFIRQIHKFEELNRTLKKMGQRP

(19) United States
(12) Patent Application Publication
(10) Pub. No.: US 2005/0233957 A1
(43) Pub. Date: Oct. 20, 2005

(54) Sodium channel regulators and modulators

(75) Inventors: Kenji Okuse, London (GB); Mark Baker, London (GB); Louisa Poon, London (GB); John Nicholas Wood, London (GB); Misbah Malik-Hall, London (GB)

Correspondence Address: NIXON & VANDERHYE, PC, 901 NORTH GLEBE ROAD, 11TH FLOOR, ARLINGTON, VA, 22203, US

(73) Assignee: University College London, London (GB)

(21) Appl. No.: 10/514,150

(22) PCT Filed: May 22, 2003
(86) PCT No.: PCT/GB03/02225

§ 371 Date: Jun. 13, 2005

(30) Foreign Application Priority Data
May 22, 2002 (GB) 0211833.9

(51) Int. Cl. G01N 33/567; G01N 33/53; C12N 5/08; A61K 38/54

(52) U.S. Cl. 514/12; 435/7.2; 435/368

(57) ABSTRACT

The present invention provides a method of identifying a modulator of a voltage gated sodium channel (VGSC), which method comprises: (a) bringing into contact a test compound, a VGSC and one or more binding partners selected from PAPIN, periaxin and HSPC025 under conditions where the VGSC and the binding partner(s) are capable of forming a complex in the absence of the test compound; and (b) measuring an activity of the VGSC, wherein a charge in the activity of the VGSC relative to the activity in the absence of the test compound indicates that the test compound is a modulator of said VGSC. Compounds identified in such screening methods are proposed for use in the treatment of VGSC-related conditions, for example in the treatment or prevention of pain. Also provided are methods of enhancing the functional expression of a voltage gated sodium channel (VGSC) in a cell comprising the step of increasing the level of a binding partner of the invention in the cell.

### FIELD OF THE INVENTION

The present invention relates generally to methods and materials for use in regulating or modulating voltage gated Na⁺ channels (VGSCs).

### BACKGROUND OF THE INVENTION

VGSCs are transmembrane proteins responsible for bestowing electrical excitability upon almost all excitable membranes. The pore is gated by depolarization of the cell membrane, transiently allowing Na+ ions to enter into the cell, and generating the upswing of an action potential. Following activation, VGSCs undergo inactivation, limiting the action potential duration, and allowing rapid membrane repolarization followed by a return to the resting state. All known VGSCs exhibit remarkable functional similarities and this is reflected in a high degree of amino-acid sequence homology. However, natural toxins are known to discriminate well between Na channel subtypes. For example, tetrodotoxin (TTX) from the Puffer fish, can selectively block subtypes of neuronal VGSCs at single nanomolar concentrations, whereas other neuronal VGSCs remain unblocked by the toxin at micromolar concentrations. These neuronal VGSCs that are TTX-insensitive or resistant (TTX-R) are found in the peripheral nervous system, and are exclusively associated with nerves involved in the transmission of pain (see e.g. Akopian et al (1999) Nature Neuroscience 2, 541-548).

WO 97/01577 (University College London) relates to a novel 1,957 amino acid TTX-insensitive VGSC from mammalian sensory neurons (which has been designated Nav 1.8). U.S. Pat. No. 6,184,349 (Syntex) discusses VGSCs. The sodium channel Nav1.8 (also known as SNS or PN3) is expressed exclusively in small diameter sensory neurones that correspond to Aδ or C-fibre nociceptors, which are the cells that transmit pain signals. One key feature of Nav1.8 pharmacology is its resistance to high concentrations of tetrodotoxin (TTX), which blocks most other sodium channels. Evidence for a role of Nav1.8 in pain signalling comes largely from knock out mice and from studies where the channel is downregulated with antisense oligonucleotides. These experiments suggest that Nav1.8 is important in models of inflammatory, neuropathic and visceral pain.

Nav1.9 (SNS2) is also found exclusively in sensory neurones that signal pain and is also resistant to TTX. The properties of the channel suggest that it is not involved in generation or propagation of action potentials but is involved in setting the level of excitability of the cell. There is evidence that G-proteins can activate Nav1.9, which in turn increases neuronal excitability and makes the cell more likely to fire. There is no direct evidence for involvement of Nav1.9 in pain models, but given its function in the cell and the restricted distribution, it could play a major role in producing the hyper-reactivity associated with many chronic pain states.

Nav 1.3 is found in brains of adult animals and is sensitive to TTX. There is normally no Nav1.3 in sensory neurones, but after nerve damage, levels are upregulated massively. Again there is no direct evidence for involvement of Nav1.3 in pain, but the selective upregulation after nerve injury suggests that it might play a role in transmission of neuropathic pain signals.

### SUMMARY OF THE INVENTION

The present invention derived from the Inventors' finding that PAPIN, periaxin and HSPC are able to act as accessory proteins, involved in the functional expression of voltage gated sodium channels (VGSCs).

The present invention provides screening methods for the identification of compounds which are capable of modulating VGSCs. In one aspect there is provided a method of identifying a modulator of a voltage gated sodium channel (VGSC), which method comprises:
(a) bringing into contact a test compound, a VGSC and one or more binding partners selected from PAPIN, periaxin and HSPC025 under conditions where the VGSC and the binding partner(s) are capable of forming a complex in the absence of the test compound; and
(b) measuring an activity of the VGSC, wherein a change in the activity of the VGSC relative to the activity in the absence of the test compound indicates that the test compound is a modulator of said VGSC.

Also within the scope of the invention are compounds identified by a method of the invention. The invention also provides the use of a compound identified by a method of the invention in the manufacture of a medicament for modulating the functional expression of a voltage gated sodium channel; and the use of an inhibitor of PAPIN, periaxin and/or HSPC025 activity or expression in the manufacture of a medicament for modulating the functional expression of a voltage gated sodium channel.

The invention also provides a method of treating a disorder or condition associated with the activity of a voltage gated sodium channel, said method comprising administering to an individual in need thereof a compound identified by a method of the invention or an inhibitor of PAPIN, periaxin and/or HSPC025 activity or expression.

The methods of the invention may be used to increase the functional expression of a VGSC such as a SNS sodium channel in the cell. The level of “functional expression” of the VGSC is used herein to describe the quantity or proportion of the VGSC which is functional on the cell membrane. Activity in this context means a capability to mediate a sodium current across a membrane in response to an appropriate stimulus.

Thus a further aspect of the present invention provides a method of enhancing the functional expression of a voltage gated sodium channel (VGSC) in a cell which method comprises the step of increasing the level of one or more binding partner(s) of the invention.

The invention also provides a host cell capable of expressing a VGSC and a binding partner selected from one or more of PAPIN, periaxin and HSPC025 wherein said VGSC and/or said binding partner is expressed from one or more heterologous expression vectors within said cell.

### BRIEF DESCRIPTION OF THE DRAWINGS

FIG. 1 shows the structure of Nav1.8 α-subunit showing the four homologous domains each of which is composed of six membrane spanning segments. FIG. 1A shows the basic structure of the subunit. The location of the three baits is indicated by arrows and the numbers correspond to the amino acid location. FIG. 1B shows the subunit in more detail.

FIG. 2 shows the map of the pEG202 plasmid, which is a yeast E. coli shuttle vector and is a multiple copy plasmid containing the yeast 2 μm origin of replication. The plasmid also contains the selectable marker genes HIS3, along with yeast promoter ADH1 gene which encodes for amino acid 1-202 of the bacterial repressor protein LexA. Bait proteins expressed from this plasmid contain amino acids 1-202 of LexA, which includes the DNA binding domain. The plasmid also contains the E. coli origin or replication and the ampicillin resistant gene. Our baits were cloned into EcoR1 and NotI sites. The numbers indicate relative map positions. FIG. 2A shows the basic structure of the plasmid, FIG. 2B provides further detail.

FIG. 3 shows in detail the various LacZ reporters which are derived from a plasmid that contains the wild-type Gal1 fused o LacZ. Reporters for measuring activation are derived from pLR1Δ1, in which the Gal1 upstream activation sequences have been inserted in place of UAS_{G} to create LacZ reporters with different sensitivities.

FIG. 4 shows yeast containing LexA-fused baits, the reporter gene and the library in pJG4-5 with a cDNA expression cassette under the control of the GAL1 promoter. This plasmid contains the TRP1 selectable marker and the 2 μm origin of replication. The numbers indicate relative map positions. FIG. 4A shows the basic structure of the plasmid, FIG. 4B provides further detail.

FIG. 5: A148 (HSPC025) allows the expression of TTX-resistant inward currents in CHO-SNS22 cells. A: High threshold TTX-resistant inward current recorded from fluorescent CHO-SNS22 cells after tranfection (lipofectamine) with GFP-A148 cDNA vector. B: average current (I/Imax)-membrane potential (Em) relation for the inward current in four CHO-SNS22 cells.

### BRIEF DESCRIPTION OF THE SEQUENCES

SEQ ID NO: 1 is the DNA sequence of the rat Nav 1.8 receptor gene and SEQ ID NO: 2 is the amino acid sequence that it encodes. These sequences are publicly available from GenBank under accession number X92184.

SEQ ID NO: 3 is the DNA sequence of the human Nav 1.8 receptor gene and SEQ ID NO: 4 is the amino acid sequence that it encodes. These sequences are publicly available from GenBank under accession number AF117907.

SEQ ID NO: 5 is the DNA sequence of the rat PAPIN gene and SEQ ID NO: 6 is the amino acid sequence that it encodes. These sequences are publicly available from GenBank under accession number NM_{—}022940.

SEQ ID NO: 7 is the DNA sequence of the rat periaxin gene and SEQ ID NO: 8 is the amino acid sequence that it encodes. These sequences are publicly available from GenBank under accession number NM_{—}023976.

SEQ BD NO: 9 is the DNA sequence of the human HSPC025 gene and SEQ ID NO: 10 is the amino acid sequence that it encodes. These sequences are publicly available from GenBank under accession number NM_{—}016091.

### DETAILED DESCRIPTION OF THE INVENTION

The present invention relates generally to screening methods for the identification of compounds capable of regulating or modulating the functional expression of sodium channels. Also provided are methods wherein such compounds are used in the treatment of conditions associated with sodium channel function, for example in the prevention or treatment of pain.

The present invention derives from the discovery that the functional expression of the TTX-insensitive voltage gated sodium channel (VGSC) Nav 1.8 (which hereinafter may be referred to as the “SNS sodium channel”) is facilitated by interaction with one or more accessory proteins. The present inventors have determined that various proteins fulfil the role of “accessory proteins” and, more specifically, that the “accessory protein” can be one, two or all of the proteins PAPIN, periaxin and/or HSPC025.

The improved function of the sodium channel appears to be effected through direct protein-protein interaction.

As described in more detail below, this interaction may be exploited, inter alia, in:
(i) enhancing the functional expression of a VGSC e.g. in cell lines which may be used for conventional modulator-screening purposes;
(ii) defining a novel target (i.e. disruption of the protein interaction site itself) for devising modulators which could lower the functional expression of the VGSC.
   Sodium Channels

The present application relates to the regulation or modulation of functional expression of sodium channels, in particular voltage gated sodium channels (VGSCs). Table 1 indicates the sequence identity between various VGSC molecules, using the rat Nav 1.8 channel as a basis for comparison:

TABLE 1
For comparison, rat 1.8 vs human 1.8 scores 83% (with gaps) or 84%
(without gaps) identity using this method. Amino acid identity was
determined over the full protein sequence. The Nav 1.8 protein
sequence was aligned with a second sequence using Clustal.
The number of identical amino acids was then scored for
each pair and divided by the total number of amino acids in the
alignment (with gaps) or the total number of aligned amino acids
(without gaps).

| Channel | Rat 1.8 | Rat 1.5 | Rat 1.9 | Rat 1.3 |
|---|---|---|---|---|
| Accession number | X92184 | M27902 | AF059030 | Y00766 |
| With gaps | 100 | 61% | 49% | 57% |
| Without gaps | 100 | 63% | 55% | 62% |

In particular, the present invention relates to VGSCs that are associated with responses to pain or are involved in pain signalling. A suitable sodium channel is preferably a VGSC that is expressed in sensory neurons. For example, a suitable VGSC may be a sensory neuron specific (SNS) VGSC, for example Nav 1.8 or Nav 1.9, or may be upregulated in sensory neurons in response to pain, for example Nav 1.3. A suitable VGSC may be tetrodotoxin (TTX) insensitive or resistant, that is, it may remain unblocked by TTX at micromolar concentrations. Generally herein the Nav 1.8 or SNS channel may be used to exemplify the invention. It will be apparent to the skilled person that references herein to Nav 1.8 or SNS sodium channels can apply equally to other VGSC and VGSC variants.

In one aspect, a VGSC for use in methods of the invention is a Nav 1.8, Nav 1.9 or Nav 1.3 channel. The nucleotide and amino acid sequences for the Nav 1.8, rat Nav 1.9 and rat Nav 1.3 channels are publicly available, for example rat sequences are available from GenBank under the accession numbers given in Table 1. The nucleotide and amino acid sequences for rat Nav 1.8 are given in SEQ ID Nos: 1 and 2 respectively and the nucleotide and amino acid sequences for human Nav 1.8 are given in SEQ ID Nos: 3 and 4 respectively.

A suitable VGSC for use in the methods of the invention may be any of these VGSCs or a species or allelic variant of any thereof. There is no requirement that the binding partner proteins (or nucleic acids) employed in the present invention have to include the full-length “authentic” sequence of the proteins as they occur in nature. A suitable VGSC may therefore also be a variant of any of these VGSCs which retains activity as a sodium channel. For example, a suitable VGSC may have greater than 65%, greater than 70%, greater than 75%, greater than 85%, greater than 95% or greater than 98% amino acid identity with any of the Nav 1.8, Nav 1.9 or Nav 1.3 sequences.

A VGSC of the invention may be any VGSC which has the ability to specifically bind a binding partner as described below. By specifically bind it is meant that the VGSC binds the binding partner preferentially to a non-binding partner peptide, for example a VGSC binds more strongly to a PAPIN, periaxin or HSPC025 peptide than to a randomly generated peptide sequence. For example, a preferred variant of the rat Nav 1.8 channel may retain all or part of one or more of the sequences defined by amino acids 893-1148, 1420-1472 and/or 1724-1844 of SEQ ID NO: 2, which are shown herein to be involved in binding to PAPIN, periaxin and HSPC025 respectively, or a species or allelic variant of these regions.

A suitable variant channel is one which retains sodium channel function. For example, a suitable variant of the Nav 1.8 sodium channel may have the normal function of a VGSC. The function of a VGSC may be measured as described below. It may also retain the tetrodotoxin insensitivity of the Nav 1.8 channel.

A suitable variant may also retain the ability to bind p11. For example, a suitable variant channel may retain the intracellular domain of a wild type VGSC. For example, a preferred variant of the rat Nav 1.8 channel may retain the N-terminal intracellular domain found at positions 1 to 127 of SEQ ID NO: 2. A suitable variant channel may have a sequence comprising amino acids 53 to 127 or amino acids 75 to 102 of SEQ ID NO: 2, which are known to be involved in binding to p11 protein, or a species or allelic variant of this region.

A suitable variant VGSC may be a fragment of a wild type VGSC or of a variant thereof as described below. A suitable fragment may be a truncated VGSC, wherein, for example, 1%, 2%, 5%, 10%, 15%, 20%, 25%, 50% or more of the original VGSC sequence has been removed. A suitable fragment may consist of or comprise a fragment of a full length VGSC, for example, 1%, 2%, 5%, 10%, 15%, 20%, 25%, 50% or more of a full length sequence. A suitable fragment may be any fragment which retains the ability to bind a binding partner of the invention. A suitable fragment may also retain the ability to function as a sodium channel. Preferably fragments represent sequences which are believed to be either unique to the channel, or are at least well conserved among VGSCs. Such a VGSC fragment may be, for example, 25 to 50, 25 to 100, 25 to 200, 25 to 500, 25 to 1000 amino acids in length or larger. Generally fragments will be at least 40, preferably at least 50, 60, 70, 80 or 100 amino acids in size.

Fragments of the proteins of the invention may be produced by any appropriate manner known in the art. Suitable methods include, but are not limited to, recombinant expression of a fragment of the DNA encoding the binding partner. Such fragments may be generated by taking DNA encoding the binding partner, identifying suitable restriction enzyme recognition sites either side of the portion to be expressed and cutting out said portion from the DNA. The portion may then be operably linked to a suitable promoter in a standard commercially available expression system. Another recombinant approach is to amplify the relevant portion of the DNA with suitable PCR primers. Small fragments of the SNS sodium channel binding partner (up to about 20 or 30 amino acids) may also be generated using peptide synthesis methods which are well known in the art.

Variants of the proteins of the invention may be generated in any suitable way known to those of skill in the art. The term “derived” includes variants produced by modification of the authentic native sequence e.g. by introducing changes into the full-length or part-length sequence, for example substitutions, insertions, and/or deletions. This may be achieved by any appropriate technique, including restriction of the sequence with an endonuclease followed by the insertion of a selected base sequence (using linkers if required) and ligation. Also possible is PCR-mediated mutagenesis using mutant primers. It may, for instance, be preferable to add in or remove restriction sites in order to facilitate further cloning. Modified sequences according to the present invention may have a sequence at least 70% identical to the sequence of the marker. Typically there would be 80% or more, 90% or more 95% or more or 98% or more identity between the modified sequence and the authentic sequence. There may be up to five, for example up to ten or up to twenty or more nucleotide deletions, insertions and/or substitutions made to the full-length or part length sequence provided functionality is not totally lost.

A suitable variant may therefore be a modified version of a naturally occurring VGSC having a different amino acid sequence. The modified version may have, for example, amino acid substitutions, deletions or additions. At least 1, at least 2, at least 3, at least 5, at least 10, at least 50, at least 100 or at least 200 amino acid substitutions or deletions, for example, may be made, up to a maximum of 1000 or 500 or 300. For example, from 1 to 1000, from 5 to 500, from 10 to 300 or from 50 to 200 amino acid substitutions or deletions may be made. Typically, if substitutions are made, the substitutions will be conservative substitutions, for example according to the following Table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The VGSC or a functional variant thereof may be fused to an additional heterologous polypeptide sequence to produce a fusion polypeptide. Thus, additional amino acid residues may be provided at, for example, one or both termini of the VGSC or a functional variant thereof. The additional sequence may perform any known function. Typically, it may be added for the purpose of providing a carrier polypeptide, by which the VGSC or functional variant thereof can be, for example, affixed to a label, solid matrix or carrier. It may often be convenient to use fusion polypeptides in the assays of the invention. This is because fusion polypeptides may be easily and cheaply produced in recombinant cell lines, for example recombinant bacterial or insect cell lines. Fusion polypeptides may be expressed at higher levels than the wild-type VGSC or functional variant thereof. Typically this is due to increased translation of the encoding RNA or decreased degradation. In addition, fusion polypeptides may be easy to identify and isolate. Typically, fusion polypeptides will comprise a polypeptide sequence as described above and a carrier or linker sequence. The carrier or linker sequence will typically be derived from a non-human, preferably a non-mammalian source, for example a bacterial source.

The VGSC or a functional variant thereof may be modified by, for example, addition of histidine residues, a T7 tag or glutathione S-transferase, to assist in its isolation. Alternatively, the heterologous sequence may, for example, promote secretion of the VGSC or functional variant thereof from a cell or target its expression to a particular subcellular location, such as the cell membrane. Amino acid carriers can be from 1 to 400 amino acids in length or more typically from 5 to 200 residues in length. The VGSC or functional variant thereof may be linked to a carrier polypeptide directly or via an intervening linker sequence. Typical amino acid residues used for linking are tyrosine, cysteine, lysine, glutamic acid or aspartic acid.

VGSCs or functional variants thereof may be chemically modified, for example, post-translationally modified. For example they may be glycosylated or comprise modified amino acid residues. They can be in a variety of forms of polypeptide derivatives, including amides and conjugates with polypeptides.

Chemically modified VGSCs or functional variants thereof also include those having one or more residues chemically derivatized by reaction of a functional side group. Such derivatized side groups include those which have been derivatized to form amine hydrochlorides, p-toluene sulfonyl groups, carbobenzoxy groups, t-butyloxycarbonyl groups, chloroacetyl groups and formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine.

Also included as chemically modified VGSCs or functional variants thereof are those which contain one or more naturally occurring amino acid derivatives of the twenty standard amino acids. For example, 4-hydroxyproline may be substituted for proline or homoserine may be substituted for serine.

In one aspect there is provided a peptide comprising at least 10, at least 15, at least 20 or at least 25 contiguous amino acids of the sequence of SEQ ID NO: 2 or SEQ ID NO: 4; or a sequence having at least 65%, at least 70%, at least 75%, at least 85%, at least 95% or at least 98% amino acid sequence identity to SEQ ID NQ: 2 or SEQ ID NO: 4, wherein said peptide is capable of specifically binding a binding partner of the invention and is less than 1000 amino acids in length. Said peptide may be for example less than 500 amino acids, less than 300 amino acids, less than 200 amino acids, less than 100 amino acids or less than 50 amino acids in length.

Similarity or identity may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wis. 53711). Preferably sequence comparisons are made using FASTA and FASTP (see Pearson & Lipman, 1988. Methods in Enzymology 183: 63-98). Parameters are preferably set, using the default matrix, as follows: Gapopen (penalty for the first residue in a gap):—16 for DNA; Gapext (penalty for additional residues in a gap):—4 for DNA KTUP word length: 6 for DNA. Alternatively, homology in this context can be judged by probing under appropriate stringency conditions. One common formula for calculating the stringency conditions required to achieve hybridization between (complementary) nucleic acid molecules of a specified sequence homology is (Sambrook et al., 1989): Tm=81.5° C.+16.6 Log [Na+]+0.41 (% G+C)−0.63 (% formamide)−600/#bp in duplex. Preferred conditions will give hybridisation of molecules at least 70% homology as described above.

The UWGCG Package provides the BESTFIT program which can be used to calculate identity (for example used on its default settings) (Devereux et al (1984) Nucleic Acids Research 12, 387-395). The PILEUP and BLAST algorithms can alternatively be used to calculate identity or line up sequences (typically on their default settings), for example as described in Altschul S. F. (1993) J Mol Evol 36:290-300; Altschul, S. F. et al (1990) J Mol Biol 215:403-10. Identity may therefore be calculated using the UWGCG package, using the BESTFIT program on its default settings. Alternatively, sequence identity can be calculated using the PILEUP or BLAST algorithms. BLAST may be used on its default settings.

Software for performing BLAST analyses is publicly available through the National Centre for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

In one aspect, a VGSC of the invention has an amino acid sequence comprising:
(a) the amino acid sequence of SEQ ID NO: 2 or SEQ ID NO: 4;
(b) a species or allelic variant of (a);
(c) a variant of (a) having at least 70% amino acid sequence identity thereto; or
(d) a fragment of any of (a) to (c).

Such a VGSC will retain the ability to bind a binding partner of the invention. Such a VGSC may also retain the ability to mediate a Na⁺ current across a membrane, such as the plasma membrane of the cell.

Sodium Channel Binding Partners

The present invention relates to the discovery that the VGSC Nav 1.8 interacts with the binding partners PAPIN, periaxin and HSPC025 protein.

PAPIN is a p0071 binding protein. p0071 is an isoform of neural plakophilin-related armadillo repeat protein (NPRAP/δ-catenin) and hence this protein has been named plakophilin-related armadillo repeat protein-interacting PSD-95/D1g-A/ZO-1 (PDZ) protein (PAPIN) (Deguchi et al 2000 J. Biol Chem 275: 29875-29880). This is a member of a family of proteins known as p120ctn, which are major substrates of tyrosine kinase phosphorylation enriched at adherens junctions, and contains 10 armadillo repeats (Reynolds, et al, 1992 Oncogene 7: 2439-2445). p120ctn directly interacts with E-cadherins. The armidillo repeat is a repeated motif of about 40 amino acids originally identified in Drosophila segment polarity gene (Hatzfeld, 1999 Int Rev Cytol 186: 179-224). The function of NPRAP/δ-catenin and p0071 is not known but since both proteins are localised at cell-cell junctions it is suggested that they play roles as components of cell-cell junctions like p120ctn (Reynolds et al, 1992, Yap et al, 1998 J Cell Biol 141: 779-789).

Periaxin was first described as a protein with a possible role in the later stages of myelination (Gillespie et al, 1994 Neuron 12: 497-508). As the myelin sheath matures, periaxin becomes more concentrated, suggesting the possibility of its role in the stabilisation of the myelin sheath. Scherer et al, (1995 Development 121: 4265-4273), have found that periaxin immunoreactivity was only detected in the Schwann cells and not the oligodendrocytes and concluded that it was only expressed in the peripheral nervous system and not the central nervous system. They also found that periaxin had similar mobility on SDS-PAGE to two proteins isolated from peripheral nerve myelin, p170 and SAG (Shuman et al, 1986 J Neurochem 47: 811-818; Dieperink et al, 1992 J Neurosci 12: 2177-2185). The authors of these papers also showed similar staining of myelinating Schwann cells to antisera against P170 as that found for staining with periaxin antiserum, and therefore concluded that they were the same proteins. Scherer et al have found that periaxin was expressed by myelinating Schwann cells, and that its localisation changes during ensheathment and myelination and therefore that it had a specific function in myelinating Schwann cells. Dytrych et al (1998 J Biol Chem 273: 5794-5800), have shown that there are two isoforms of periaxin, L-periaxin and S-periaxin Both proteins have an N-terminal PDZ protein binding domains. L-periaxin also possesses a tripartite (three basic sequences) nuclear localisation sequence (NLS) (Shermann et al, 2000 J Biol Chem 275: 4537-4540). NLS are short sequences that have the capacity to transport heterologous proteins into the nucleus (Nigg, 1997 Nature 386: 779-787). Shermann et al have shown that the NLS also localises L-periaxin to the Schwann cell nucleus when it is first expressed in the embryonic PNS and that it is subsequently localised to the plasma membrane.

HSPC025 appears, in a homology search, to have some sequence related to the G-protein coupled receptor for the protein rhodopsin found in the eye. Stimulation of G-protein coupled receptors (GPCRs) by hormones, growth factors, neurotransmitters and sensory stimuli may result in an increase in intracellular calcium, cyclic AMP, or a variety of other intracellular second messages.

As any one of PAPIN, periaxin or HSPC025 may be used interchangeably in the methods or compositions of the invention, for ease of reference the term “binding partner” shall be used generically from hereon in to describe one or more of the three proteins, or a variant of any thereof.

Binding partners of the invention, including PAPIN, periaxin and HSPC025 may be obtained either from publicly available sources or using known procedures. Specifically, they may be obtained by reference to the GENBANK or EMBL databases. For example, rat PAPIN DNA has the GENBANK accession number NM 022940 (SEQ ID NO: 5) and rat PAPIN protein has the GENBANK accession number NP 075229 (SEQ ID NO: 6); and rat periaxin DNA has the GENBANK accession number NM 023976 (SEQ ID NO: 7) and rat periaxin protein has the GENBANK accession number NP 076466 (SEQ ID NO: 8). Human HSPC025 (also known as EIP3S6IP—eukaryotic translation initiation factor 2, subunit 6 interacting protein) DNA has the GENBANK accession number NM 016091 (SEQ ID NO: 9) and human HSPC025 protein has the GENBANK accession number NP 057175 (SEQ ID NO: 10). Mouse clones RAF67 (a 67 kDa polymerase-associated factor) and HSP-66Y (tyrosine-rich heat shock protein) have 92% homology to the HSPC025 clone described herein. The mouse clone RAF67 may be obtained under GENBANK accession numbers AJ310346 (DNA) and CAC84554 (protein) and clone HSP-66Y may be obtained under GENBANK accession numbers AB066095 (DNA) and BAB85122 (protein).

According to the present invention, a suitable binding partner for use in the present invention may be a naturally occurring binding partner peptide, or may be an artificially constructed binding partner. A suitable binding partner may be a full-length binding partner protein or a species or allelic variant thereof. For example, a suitable binding partner may have the amino acid sequence of rat PAPIN given in SEQ ID NO: 6, the amino acid sequence of rat periaxin given in SEQ ID NO:8 or the amino acid sequence of human HSPC025 given in SEQ ID NO: 10. A suitable binding partner may alternatively be a species or allelic variant of the polypeptides of SEQ ID Nos: 6, 8 or 10.

There is no requirement that the binding partner proteins (or nucleic acids) employed in the present invention have to include the full-length “authentic” sequence of the binding partner proteins as they occurs in nature. Variants may be used (e.g. which are derived from the sequences of SEQ ID Nos 6, 8 or 10 for example) which retain the ability to modify the functional expression of a VGSC, for example the ability of a VGSC to mediate a sodium current through a membrane.

Modified binding partner sequences according to the present invention may have an amino acid sequence at least 70% identical to the sequence of an endogenous binding partner such as the rat PAPIN of SEQ ID NO: 6, the rat periaxin of SEQ ID NO: 8 or the human HSPC025 of SEQ ID NO: 10. Typically there would be 75% or more, 85% or more 95% or more, 98% or more or 99% or more identity between the modified sequence and the authentic sequence, for example a naturally occurring sequence. Sequence identity can be calculated using the methods described above. The BESTFIT program of the UWGCG package may be used on its default settings. Alternatively, the PILEUP pr BLAST algorithms may be used on their default settings.

A functional variant may be a modified version of a binding partner, for example a modified version of a naturally occurring PAPIN, periaxin or HSPC025 polypeptide. Such a modified version may have, for example, amino acid substitutions, deletions or additions. Such substitutions, deletions or additions may be made, for example, to the sequences of rat PAPIN, rat periaxin or human HSPC025 given in SEQ ID Nos: 6, 8 and 10 respectively. Any deletions, additions or substitutions must still allow the binding partner to bind to a VGSC and preferably will allow the binding partner to enhance the functional expression of the VGSC as described herein. At least 1, at least 2, at least 3, at least 5, at least 10, at least 20 or at least 50 amino acid substitutions or deletions, for example, may be made up to a maximum of 70 or 50 or 30. For example, from 1 to 70, from 2 to 50, from 3 to 30 or from 5 to 20 amino acid substitutions or deletions may be made. Typically, if substitutions are made, the substitutions will be conservative substitutions as described above. Deletions are preferably deletions of amino acids from regions not involved with the interaction with VGSCs.

A binding partner or a functional variant thereof may be fused to an additional heterologous polypeptide sequence to produce a fusion polypeptide, as long as the binding partner is still capable of binding a VGSC. Such a fusion polypeptide may be a carrier polypeptide or contain a linker sequence. Such polypeptides are described above.

The binding partners and functional variants thereof of the invention may be chemically modified as described above.

A suitable variant binding partner may be a fragment of a wild type binding partner or of a variant thereof as described above. A suitable fragment may be a truncated binding partner, wherein, for example, 1%, 2%, 5%, 10%, 15%, 20%, 25%, 50% or more of the original binding partner sequence has been removed. A suitable fragment may consist of or comprise a fragment of a full length binding partner, for example, 1%, 2%, 5%, 10%, 15%, 20%, 25%, 50% or more of a full length sequence. A suitable fragment may be any fragment which retains the ability to bind a VGSC. A fragment may be, for example, 5, 10, 15, 20, 25, 30, 40, 50, 60, 70 80, 90 or more amino acids in length.

A suitable binding partner may comprise a fragment of a wild-type or variant binding partner sequence as part of its amino acid sequence. Such a variant will retain the ability to bind VGSC.

A PAPIN fragment which retains the ability to bind VGSC may consist of or comprise the C-terminal 201 amino acids (amino acids 2566 to 2766) of SEQ ID NO: 6, or the C-terminal 210 amino acids (amino acids 2557 to 2766) of SEQ ID NO: 6. Such a PAPIN fragment may be, for example, 201 to 500, 201 to 1000, 201 to 1500 amino acids in length or larger. Alternatively, such a fragment may be or comprise a fragment of the sequence from amino acid 2566 to amino acid 2766 of SEQ ID NO: 6, which retains the ability to bind VGSC. Such a fragment may be, for example, 20, 50, 100, 150, 200 or more amino acids in length or larger. A suitable PAPIN may be a C-terminal fragment of a naturally occurring or variant PAPIN protein. A PAPIN fragment which retains the ability to bind a VGSC may consist of or comprise the two PDZ domains of PAPIN that lie closest to the C-terminus of the full length protein. Such a fragment may comprise further regions or domains of the PAPIN protein that lie close to these PDZ domains in the full length naturally occurring protein. A suitable PAPIN fragment may comprise an equivalent fragment to those described herein derived from the sequence of a variant PAPIN sequence, for example an allelic or species variant, or a variant as described herein which retains the ability to bind VGSC.

A periaxin fragment which retains the ability to bind VGSC may consist of or comprise the C-terminal 482 amino acids (amino acids 902 to 1383) of SEQ ID NO: 8. Alternatively, such a periaxin fragment may be or comprise a fragment of the sequence from amino acid 902 to amino acid 1383 to SEQ ID NO: 8 which retains the ability to bind VGSC. Such a fragment may be, for example, 20, 50, 100, 150, 200, 300, 400 or more amino acids in length or larger. Such a fragment may be, for example, 482 to 500, 482 to 1000, 482 to 1500 amino acids in length or larger. A suitable periaxin fragment may be a C-terminal fragment of a naturally accurring periaxin protein. A suitable periaxin fragment may comprise an equivalent fragment of those described herein, derived from a variant periaxin sequence, for example an allelic or species variant, or a variant as described herein that retains the ability to bind VGSC.

A HSPC025 fragment which retains the ability to bind VGSC may be, for example, 20 to 100, 50 to 200, 50 to 300, 50 to 400 or 50 to 500 amino acids in length or larger. Such a fragment may be a N-terminal fragment of a naturally occurring or variant HSPC025 protein.

A PAPIN polypeptide for use in the methods of the present invention may therefore have an amino acid sequence comprising:
(a) the amino acid sequence of SEQ D NO: 6;
(b) a species or allelic variant of (a);
(c) a variant of (a) having at least 70% amino acid sequence identity thereto; or
(d) a fragment of any of (a) to (c).

Such a PAPIN peptide will retain the ability to bind a VGSC.

A periaxin polypeptide for use in the methods of the present invention may therefore have an amino acid sequence comprising:
(e) the amino acid sequence of SEQ ID NO: 8;
(f) a species or allelic variant of (a);
(g) a variant of (a) having at least 70% amino acid sequence identity thereto; or
(h) a fragment of any of (a) to (c).

Such a periaxin peptide will retain the ability to bind a VGSC.

A HSPC025 polypeptide for use in the methods of the present invention may therefore have an amino acid sequence comprising:
(i) the amino acid sequence of SEQ ID NO: 10;
(j) a species or allelic variant of (a);
(k) a variant of (a) having at least 70% amino acid sequence identity thereto; or
(l) a fragment of any of (a) to (c).

Such a HSPC025 peptide will retain the ability to bind a VGSC.

The term “derived” includes variants produced by modification of the authentic native sequence e.g. by introducing changes into the full-length or part-length sequence, for example substitutions, insertions, and/or deletions. This may be achieved by any appropriate technique, for example as described above.

As described in more detail below, the level of SNS sodium channel binding partner expression in the cell will generally be increased by introducing it into the cells by causing or allowing expression from heterologous nucleic acid encoding therefor.

Nucleic Acids

The present invention also encompasses the use of nucleic acids which encode VGSCs or binding partners of the invention to produce such proteins. For example, provided in the sequence listing are nucleic acid sequences encoding the rat Nav 1.8 channel (SEQ ID NO: 1), the human Nav 1.8 channel (SEQ ID NO: 3), rat PAPIN (SEQ ID NO: 5), rat periaxin (SEQ ID NO: 7) and human HSPC025 (SEQ ID NO: 9). Test compounds for use in the assay methods of the invention may also be nucleic acids or may be provided as nucleic acids which encode a test polypeptide.

Generally, nucleic acids, for example heterologous nucleic acids of, or for use in, the present invention (e.g. encoding a binding partner or VGSC of the invention) may be provided isolated and/or purified from their natural environment, in substantially pure or homogeneous form, or free or substantially free of other nucleic acids of the species of origin. Where used herein, the term “isolated” encompasses all of these possibilities. Nucleic acid according to the present invention may be in the form of, or derived from, cDNA, RNA, genomic DNA and modified nucleic acids or nucleic acid analogues.

Thus the invention also relates, in a further aspect, to use of a heterologous nucleic acid molecule which comprises a nucleotide sequence encoding an SNS sodium channel binding partner described above, in the various methods of the invention.

The term “heterologous” is used broadly herein to indicate that the gene/sequence of nucleotides in question (e.g. encoding a binding partner or VGSC of the invention) have been introduced into said cells using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a cell may be non-naturally occurring in cells of that type, variety or species.

Nucleic acid sequences which encode a polypeptide in accordance with the present invention can be readily prepared by the skilled person using the information and references contained herein and techniques known in the art (for example, see Sambrook, Fritsch and Maniatis, “Molecular Cloning, A Laboratory Manual”, Cold Spring Harbor Laboratory Press, 1989, and Ausubel et al., Short Protocols in Molecular Biology, John Wiley and Sons, 1992). These techniques include (i) the use of the polymerase chain reaction (PCR) to amplify samples of the relevant nucleic acid, e.g. from genomic sources, (ii) chemical synthesis, or (iii) preparation of cDNA sequences.

Constructs and Vectors

In cell-based assay embodiments of the present invention, the polypeptide(s) of interest can be introduced into a cell by causing or allowing the expression in the cell of an expression construct or vector.

The construct may include any other regulatory sequences or structural elements as would commonly be included in such a system, and as is described below. The vector components will usually include, but are not limited to, one or more of an origin of replication, one or more marker genes, an enhancer element, a promoter, and a transcription termination sequence. Construction of suitable vectors containing one or more of these components employs standard ligation techniques which are known to the skilled artisan. Nucleic acid sequences which enable a vector to replicate in one or more selected host cells are well known for a variety of bacteria, yeast, and viruses. For example, various viral origins (SV40, polyoma, adenovirus, VSV or BPV) are useful for cloning vectors in mammalian cells.

Particularly preferred for use herein is an expression vector e.g. in the form of a plasmid, cosmid, viral particle, phage, or any other suitable vector or construct which can be taken up by a cell and used to express a coding sequence. Expression vectors usually contain a promoter which is operably linked to the protein-encoding nucleic acid sequence of interest, so as to direct mRNA synthesis. Promoters recognized by a variety of potential host cells are well known. “Operably linked” means joined as part of the same nucleic acid molecule, suitably positioned and oriented for transcription to be initiated from the promoter. DNA operably linked to a promoter is “under transcriptional control” of the promoter. Transcription from vectors in mammalian host cells is controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g. the actin promoter or an immunoglobulin promoter, and from heat-shock promoters, provided such promoters are compatible with the host cell systems.

Expression vectors of the invention may also contain one or more selection genes. Typical selection genes encode proteins that (a) confer resistance to antibiotics or other toxins e.g. ampicillin, neomycin, methotrexate, or tetracycline, (b) complement auxotrophic deficiencies, or (c) supply critical nutrients not available from complex media, e.g., the gene encoding D-alanine racemase for Bacilli. The protein encoding sequences may include reporter genes which may be any suitable reporter gene used in the art. Such reporter genes includes chloramphenicol acetyl transferase (CAT), β-galactosidase, luciferase or GFP.

Where a cell line is used in which more than one polypeptide of the invention, for example both the VGSC and binding partner, or more than one binding partner, are heterologous, these proteins may be expressed from a single vector or from two separate vectors. More than one copy of the protein encoding sequences may be present in the vector.

Cells

The methods referred to above may therefore further include introducing a nucleic acid into a host cell. The introduction, which may be generally referred to without limitation as “transformation”, may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For example, the calcium phosphate precipitation method of Graham and van der Eb, Virology 52:456-457 (1978) can be employed. General aspects of mammalian cell host system transformations have been described in U.S. Pat. No. 4,399,216. For various techniques for transforming mammalian cells, see Keown et al., Methods in Enzymology, 18-5:527 537 (1990) and Mansour et al., Nature 336:348-352 (1988).

The cells used in methods of the present invention may be present in, or extracted from, organisms. The methods of the invention may also be carried out in cells or cell lines transiently or permanently transfected or transformed with the appropriate proteins or nucleic acids encoding them. The term “in vivo” where used herein includes all of these possibilities. Thus in vivo methods may be performed in a suitably responsive cell line which expresses the VGSC (either as a native channel, or from a vector introduced into the cell). The cell line may be in tissue culture or may be a cell line xenograft in a non-human animal subject.

The host cell may express:
a VGSC and a binding partner of the invention,
a VGSC and more than one binding partner of the invention, or
a VGSC, one or more binding partners of the invention and p11.

Where a cell expresses more than one binding partner of the invention, the binding partners may be related, for example naturally occurring PAPIN and one or more PAPIN variants as described above, naturally occurring periaxin and one or more periaxin variants as described above, or naturally occurring HSPC025 and one or more variants of HSPC025 as described above. Alternatively, one or more related variants may be expressed, in the absence of a naturally occurring binding partner, for example one or more PAPIN variants as described above, one or more periaxin variants as described above or one or more variants of HSPC025 as described above.

Alternatively, the cell may express one or more unrelated binding partners, for example the cell may express PAPIN or a varient thereof with periaxin or a variant thereof; PAPIN or a variant thereof with HSPC025 or a variant thereof; periaxin or a variant thereof with HSPC025 or a variant thereof; or PAPIN or a variant thereof, periaxin or a variant thereof and HSPC025 or a variant thereof. Any combination of binding partners and/or binding partner variants described herein may be expressed in a cell of the invention or used in an assay of the invention.

In the embodiments described herein, a cell of the invention may also express p11 or a variant thereof capable of binding a VGSC, and assays of the invention may be carried out in the presence of such a p11 peptide. A suitable p11 peptide may be, for example, the rat p11 gene having a sequence available from GenBank under accession number J03627, or the human p11 gene having a sequence available from GenBank under accession number NM_{—}002966. A suitable p11 peptide may be a variant or fragment of either of these sequences that retains the ability to bind a VGSC.

The level of binding partner and/or VGSC expression in a cell may be increased by introducing it into the cells directly or by causing or allowing expression from heterologous or endogenous nucleic acid encoding therefore. The present invention therefore encompasses cells which express VGSC and one or more binding partners according to the present invention, one or more of which may be heterologously expressed.

A cell may be used which endogenously expresses binding partner and/or VGSC without the introduction of heterologous genes. That is, the VGSC and/or one or more binding partners may be endogenously expressed within the cell from the cell's own genome. Such a cell may endogenously express sufficient levels of binding partner and/or VGSC for use in the methods of the invention, or may express only low levels of binding partner and/or VGSC which require supplementation as described herein.

The assays of the invention may be carried out in a cell that endogenously expresses a VGSC and one or more binding partners of the invention. The present invention also encompasses cells in which one or more components is heterologous. For example, a cell may endogenously express a VGSC and may be stimulated to express (e.g. by transfection with a suitable vector) one or more binding partners of the invention. A cell may endogenously express one or more binding partners of the invention and may be stimulated to express a VGSC and optionally one or more further binding partners of the invention. Alternatively, a cell may be used which endogenously expresses no binding partner or VGSC, but which can be made to express binding partner(s) and VGSC using methods such as those described herein.

Heterologous expression may be achieved by transfection with a vector as described above that allows expression of one or more polypeptides of the invention (for example a VGSC and/or one or more binding partners), or may be achieved by activating one or more endogenous genes in the cell.

For example, expression of an endogenous gene may be upregulated artificially. This may be achieved by methods known in the art, for example by targeting one or more transcription factors to bind to the desired gene(s), e.g. a VGSC or binding partner gene, in the genome of the cell. Suitable transcription factors may comprise a domain capable of binding specifically to the gene of interest, e.g. a zinc finger domain, and a functional domain that can regulate expression of the gene. Such a transcription factor may be introduced into a cell as a protein or may be expressed from encoding DNA introduced into a cell. Suitable transcription factors may be generated using the ZFP technology of Sangamo BioSciences, Inc. (www.sangamo.com).

A cell may also be derived from a cell in which expression has been stimulated as described herein, for example by culturing such a cell and allowing it to proliferate. A suitable cell may also be a cell fusion comprising a cell of the invention that has been fused with a different cell type.

In the cells of the invention, said VGSC and said binding partner(s) should be expressed such that the binding partner(s) interacts to upregulate the functional expression of the VGSC. Such host cells are suitable for use in the screening methods of the invention.

The cell lines used in assays of the invention may be used to achieve transient expression of a binding partner or VGSC of the invention, although in a further aspect of the invention cells which are stably transfected with constructs which express a binding partner of the invention and, where required, a VGSC may also be generated. Means to generate stably transformed cell lines are well known in the art and such means may be used here. Preferred cells are non-neuronal e.g. CHO cells.

Host cells transfected or transformed with expression or cloning vectors described herein may be cultured in conventional nutrient media. The culture conditions, such as media, temperature, pH and the like, can be selected by the skilled artisan without undue experimentation. In general, principles, protocols, and practical techniques for maximizing the productivity of cell cultures can be found in “Mammalian Cell Biotechnology: a Practical Approach”, M. Butler, ed. JRL Press, (1991) and Sambrook et al, supra.

Transgenic Organisms

As stated above, host cells according to the present invention (for example including a heterologous binding partner for increasing VGSC expression) may be comprised in a transgenic animal, and the present invention further provides uses of the transgenic animal in the methods herein. The transgenic organisms of the invention all include within a plurality of their cells a cloned recombinant or synthetic DNA sequence which encodes, for example, a heterologous binding partner of the invention.

For more details regarding the production of transgenic organisms, and specifically transgenic mice, refer to U.S. Pat. No. 4,873,191, issued Oct. 10, 1989 (incorporated herein by reference to disclose methods producing transgenic mice), and to the numerous scientific publications referred to and cited therein.

Increasing Functional VGSC Expression

The foregoing discussion has been generally concerned with uses of the nucleic acids of the present invention for production of functional polypeptides, thereby increasing the concentration of a binding partner in a cell so as to increase functional expression of the sodium channel.

The present invention provides a method for enhancing the functional expression of a VGSC comprising exposing said channel to a binding partner of the invention. Thus the invention provides a method of modifying the translocation of a voltage gated sodium channel into a plasma membrane of a cell, which method comprises the step of altering the concentration of one or more binding partners of the invention in the cell.

Such a method may be used to increase the functional expression of a VGSC in the cell. The level of “functional expression” of the channel is used herein to describe the quantity or proportion of the channel which is active within a cell.

“Active” in this context means capable of mediating a sodium current across a membrane in response to an appropriate stimulus.

Thus a further aspect of the present invention provides a method of enhancing the functional expression of a VGSC in a cell, which method comprises the step of increasing the level of one or more binding partners of the invention in the cell.

The VGSC may be any VGSC of the invention as described above. The binding partner(s) may be any binding partner(s) of the invention as described above. The cell may be any suitable cell line as described above. Preferably the VGSC is expressed within the cell. The binding partner may also be expressed within the cell or may be applied to the cell. The VGSC and/or the binding partner(s) may be expressed from endogenous genes within the cell or from heterologous genes that have been introduced into the cell, for example by transfection of the cell with one or more vectors as described above.

Preferably, a binding partner of the invention is either applied to the cell or is heterologously expressed within the cell. The binding partner(s) may be expressed under the control of an inducible promoter so that the level of binding partner expressed within the cell may be regulated By heterologously providing binding partner(s) to the cell, the functional expression of the VGSC, that is the recruitment of the VGSC to the membrane and the subsequent activity of the VGSC, may be enhanced.

A cell in which the functional expression of a VGSC has been enhanced by such a method may be subsequently used in a screening method of the invention. Such a cell will have enhanced VGSC functional expression and will therefore be particularly sensitive to any changes in VGSC activity that a test compound may cause.

The information disclosed herein may also be used to reduce the activity of a binding partner in cells in which it is desired to do so, with a corresponding reduction in the functional expression of the sodium channel.

For instance down-regulation of expression of a target gene may be achieved using anti-sense technology.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Smith et al, (1988) Nature 334,724-726. Such methods would use a nucleotide sequence which is complementary to the coding sequence. Further options for down regulation of gene expression include the use of ribozymes, e.g. hammerhead ribozymes, which can catalyse the site-specific cleavage of RNA, such as mRNA (see e.g. Jaeger (1997) The new world of ribozymes, Curr Opin Struct Biol 7:324-335, or Gibson & Shillitoe (1997) Ribozymes: their functions and strategies form their use, Mol Biotechnol 7: 242-251.)

As is demonstrated in the Examples hereinafter, the binding partners of the present invention demonstrate particular efficacy in the down-regulation of expression of a VGSC, particularly an SNS sodium channel. In cultured dorsal root ganglia the activity of the SNS sodium channel is determined by measurement of the current across the channel. In the antisense experiment described in the Examples, PAPIN resulted in a 75% (n=8) inhibition of that current, Periaxin resulted in a 61% (n-11) inhibition and HSPC025 resulted in a 62% (n=9) inhibition of the current. These results indicate that the binding partners of the present invention are of particular interest in the modulation of the SNS sodium channel(s). The present invention therefore also relates to the use of a binding partner in the down-regulation of expression of a VGSC such as an SNS sodium channel.

Assays Using Enhanced VGSC Functional Expression

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility.

One aspect of the present of the present invention provides assays having enhanced sensitivity utilising the enhanced sodium channel functionality which can be achieved using a binding partner as hereinabove defined. Such systems (e.g. cell lines) are particularly useful for identifying compounds capable of modulating a VGSC such as the SNS sodium channel.

“Modulating” includes blocking or inhibiting the activity of the channel in the presence of, or in response to, an appropriate stimulator. Alternatively modulators may enhance the activity of the channel. Preferred modulators are channel blockers or inhibitors.

The screening methods described herein generally assess whether a test compound or putative modulator are capable of causing a change in an activity of a VGSC. Any activity normally exhibited by a VGSC may be measured. For example, a suitable activity may be the ability of the VGSC to bind specifically to or to form a complex with a binding partner of the present invention. Such a binding activity may be measured using methods known in the art, such as those described herein. A test compound which modulates this binding activity is a potential modulator of VGSC. Another activity of VGSCs which may be measured is the ability to function as a sodium channel. This may be measured using methods known in the art such as those described herein. For example, a test compound may affect the ability of a VGSC to produce a sodium current across a membrane in which the VGSC is present. Such assays may include the application of a specific stimulus, for example a stimulus which would normally result in sodium current flow.

This aspect of the invention may take the form of any, preferably in vivo, assay utilising the enhanced sodium channel functionality which can be achieved using a binding partner of the invention such as PAPIN, periaxin or HSPC025. The term “in vivo” includes cell lines and the like as described above. Thus the in vivo assays may be performed in a suitably responsive cell line which expresses a VGSC such as the SNS sodium channel (either as a native channel, or from a vector introduced into the cell) and a heterologous or endogenous binding partner. The cell line may also express p11 as described above. In the in vivo assays of the invention, it will be desirable to achieve sufficient expression of a binding partner to recruit a VGSC such as an SNS sodium channel to the membrane to enhance its functional expression. However, the precise format of the assays of the invention may be varied by those of skill in the art using routine skill and knowledge.

Thus the invention provides methods of modulating a VGSC, the functional expression of which has been enhanced, which method comprises the step of contacting said channel with a putative modulator thereof.

The contacting step may be in vivo or in vitro, as described in more detail below. One suitable system for testing modulation (e.g. inhibition or blockage) of a VGSC, is the CHO-SNS employed in the Examples below. Other systems are disclosed e.g. in WO 97/01577. Membrane currents are conveniently measured with the whole-cell configuration of the patch clamp method, according to the procedure detailed in the Examples. Preferred voltage clamps are those in which the cell potential is stepped from the holding potential of about −90 mV to test potentials that range from about −110 mV to +60 to 80 mV. In order to isolate TTX-R sodium currents, TTX, 4-aminopyridine (AP) and CdC12 were used with tetraethyl ammonium ions (TEA), and Cs. However those skilled in the art will be aware of other such compounds and combinations of compounds which could be used analogously.

In one embodiment these is provided a method for identifying a modulator of a VGSC which method comprises the steps of:
(i) providing a cell in which the functional activity of said channel has been enhanced as described above (e.g. by increasing the concentration of a sodium channel binding partner in the cell e.g. by causing or allowing expression from a nucleic acid encoding a binding partner of the invention in the cell);
(ii) contacting (directly or indirectly) the channel in the cell with the test compound;
(iii) measuring the activity (e.g. the current mediated by the channel, optionally in the presence of an activator) of the channel.

Preferably the activity before and after the contacting with the test compound will be compared, and optionally the relative activity will be correlated with the modulatory activity of the test compound. Compounds may therefore be identified that are capable of modulating the activity of a VGSC. Such compounds may have therapeutic use in the treatment or prevention of conditions associated with VGSC activity as described in more detail below.

Methods of the present invention may be employed in high throughput screens analogous to those well known in the art—see e.g. WO 200016231 (Navicyte); WO 200014540 (Tibotec); DE 19840545 (Jerini Biotools); WO 200012755 (Higher Council for Scientific Research); WO 200012705 (Pausch M H; Wess J); WO 200011216 (Bristol-Myers Squibb); U.S. Pat. No. 6,027,873 (Genencor Intl.); DE 19835071 (Carl Zeiss; F Hoffman-La Roche); WO 200003805 (CombiChem); WO 200002899 (Biocept); WO 200002045 (Euroscreen); U.S. Pat. No. 6,007,690 (Aclara Biosciences).

Compounds (putative sodium channel modulators) which may be used may be natural or synthetic chemical compounds used in drug screening programmes. Extracts of plants which contain several characterised or uncharacterised components may also be used. In preferred embodiments the substances may be provided e.g. as the product of a combinatorial library such as are now well known in the art (see e.g. Newton (1997) Expert Opinion Therapeutic Patents, 7(10): 1183-1194). The amount of putative modulator compound which may be added to an assay of the invention will normally be determined by trial and error depending upon the type of compound used. Typically, from about 0.01 to 100 nM concentrations of putative modulator compound may be used, for example from 0.1 to 10 nM. Modulator compounds may be those which either agonise or antagonise the interaction. Antagonists (inhibitors) of the interaction are particularly desirable.

Interaction Between Binding Partner and Sodium Channel

The interaction of a binding partner, such as hereinabove defined, and a VGSC such as an SNS sodium channel, may be investigated, optionally using fragments of one or both proteins. The proteins or fragments may be labelled to facilitate this.

For example the proteins or fragments can be linked to a coupling partner, e.g. a label. Techniques for coupling labels to peptidyl coupling partners are well known in the art. Labels may be fluorescent marker compounds expressed as fusions e.g. GFP. In another embodiment the proteins or fragments may be radiolabelled. Radiolabelling of peptides can be achieved using various methods known in the art. For example, peptides can be labelled with a radioactive isotope through use of a chelating agent or by covalent labelling with a material capable of direct reaction with a peptide (such as iodine), as well as by direct labelling (substitution of a radioactive isotope, such as ¹⁴C or tritium, for an atom present in the peptide) or ³⁵S-methionine which may be incorporated into recombinantly produced proteins. Generally, radiolabelled peptides containing tyrosine will be prepared using ¹²⁵I, or by tritium exchange. See U.S. Pat. No. 5,384,113, as well as numerous other patent and other publications, for general techniques available for the radiolabelling process. As used herein, the term “radiolabeled” describes a product that has been attached to a radioisotope by any of the various known methods, such as by covalent labelling or covalent binding, by a direct substitution method, or by a chelation method.

Other suitable detectable labels include tags such as an HA tag, GST or histidine. Recombinantly produced protein may also be expressed as a fusion protein containing an epitope which can be labelled with an antibody. Alternatively, an antibody against the proteins can be obtained using conventional methodology.

In a further aspect of the invention, the labelling methods described above are used to identify the binding site on a VGSC for a binding partner (and vice versa). Such methods will generally comprise the steps of producing a fragment of one or both proteins, and contacting said fragment with its binding partner (all or part of it) and determining whether binding occurs. Preferably one or both partners will be labelled and/or tagged to facilitate the detection of binding.

For example, in order to identify the binding site for a binding partner such as is hereinabove defined in a domain of a VGSC such as an SNS ion channel, small segments of the domain believed to contain said binding site may be tested.

Preferred fragments may be selected from a domain of the Nav 1.8 ion channel. Preferably fragments represent sequences which are believed to be either unique to the VGSC, or are at least well conserved among voltage-gated sodium channels.

Preferred fragments include amino acid positions 893-1148, 1420-1472 and/or 1723-1844 (numbered according to the rat Nav 1.8 sodium channel sequence of SEQ ID NO: 2).

Binding fragments can be identified using the GST “pull down assay”. Briefly, protein, for example a PAPIN, periaxin or HSPC025 protein, produced in COS-7 cells by lipofection is mixed with fragments of a VGSC, for example fragments as described above which are fused to GST made in bacteria. These protein complexes are collected by glutathione beads and the protein is recovered only when the VGSC fragment has one or more binding site(s) for it. In other embodiments, co-immunoprecipitation or an overlay assay can be done in place or in addition to the “pull down” assay.

The binding site can be further investigated e.g. using point mutations by recombinant PCR or a uracil containing vector system (Fitzgerald et al 1999 J Physiology 516.2, 433446). Since the target cDNA (e.g. corresponding to a fragment described above of a VGSC domain may be fairly short, recombinant PCR may be preferred. Mutated fragments may again be tested e.g. in the GST “pull down” assay, to precisely identify the interaction site between the VGSC and the binding partner.

Once identified the binding site may be modelled in 3 dimensions to produce mimetics. Alternatively it may be used directly e.g. as a binding partner (optionally in phage display) to screen for compounds.

Assay for Modulators of Interaction

In a further aspect the present invention provides an assay for a modulator of the functional expression of a VGSC in a cell, which assay comprises the steps of:
(a) bringing into contact a VGSC, one or more binding partners, and a putative modulator compound under conditions where the VGSC and the binding partner(s), in the absence of modulator, are capable of forming a complex; and
(b) measuring the degree of inhibition of complex formation caused by said modulator compound.

The present invention further provides an assay for a modulator of the functional expression of VGSC in a cell, which assay comprises the steps of:
(a) bringing into contact a VGSC, one or more binding partners, and a putative modulator compound under conditions where the VGSC and the binding partner(s), in the absence of modulator, are capable of forming a complex; and
(b) exposing the VGSC to a stimulus such as to produce to a sodium current across a membrane in which the VGSC is present;
(c) measuring the degree of inhibition of the current caused by said modulator compound.

One assay format which is widely used in the art to study the interaction of two proteins is a two-hybrid assay. This assay may be adapted for use in the present invention. A two-hybrid assay comprises the expression in a host cell of the two proteins, one being a fusion protein comprising a DNA binding domain (DBD), such as the yeast GAL4 binding domain, and the other being a fusion protein comprising an activation domain, such as that from GAL4 or VP16. In such a case the host cell (which may be bacterial, yeast, insect or mammalian, particularly yeast or mammalian) will carry a reporter gene construct with a promoter comprising a DNA binding elements compatible with the DBD. The reporter gene may be a reporter gene such as chloramphenicol acetyl transferase, luciferase, green fluorescent protein (GFP) and β-galactosidase, with luciferase being particularly preferred.

Two-hybrid assays may be in accordance with those disclosed by Fields and Song, (1989, Nature 340; 245-246). In such an assay the DNA binding domain (DBD) and the transcriptional activation domain (TAD) of the yeast GAL4 transcription factor are fused to the first and second molecules respectively whose interaction is to be investigated. A functional GAL4 transcription factor is restored only when two molecules of interest interact. Thus, interaction of the molecules may be measured by the use of a reporter gene operably linked to a GAL4 DNA binding site which is capable of activating transcription of said reporter gene.

Thus two hybrid assays may be performed in the presence of a potential modulator compound and the effect of the modulator will be reflected in the change in transcription level of the reporter gene construct compared to the transcription level in the absence of a modulator.

Host cells in which the two-hybrid assay may be conducted include mammalian, insect and yeast cells, with yeast cells (such as S. cerivisiae and S. pombe) being particularly preferred.

The interaction between a binding partner and a VGSC may also be assessed in mammalian cells. Cells or cell lines are derived which (over) express the VGSC in a zero binding partner background or in the background of endogenously expressed binding partner or in the background of (over)expressed binding partner. This can be done by (co)transfecting the VGSC with or without binding partner into the cell. Any cell may be chosen and VGSC expression and/or binding partner expression may be transient or stable. The effect of binding partner on the VGSC can be determined by comparing ion flux across the channel in cells (over)expressing binding partner with those that do not (over)express binding partner or show low levels of binding partner expression. Other ways of measuring the effect of binding partner on the VGSC are by assaying the extent of membrane localisation of the VGSC in whole cells or in isolated membranes. VGSC localisation can be assessed by antibody staining in cellular immunofluorescence assays, or by western blotting of membrane fractions or by toxin binding on whole cells or membrane fractions. The interaction can also be derived in co-immunoprecipitation assays of binding partner and VGSC. Inhibitors of the interaction will inhibit the functionality or the membrane localisation of VGSC, or the extent of co-immunoprecipitation between binding partner and VGSC in the cells (over)expressing binding partner.

Another assay format measures directly, in vivo or in vitro the interaction between a binding partner and a VGSC by labelling one of these proteins with a detectable label (see above) and bringing it into contact with the other protein which has been optionally immobilised on a solid support, either prior to or after proteins have been brought into contact with each other.

The protein which is optionally immobilized on a solid support may be immobilized using an antibody against that protein bound to a solid support or via other technologies which are known per se. In the Examples which follow a preferred in vitro interaction is illustrated which utilises a fusion protein of the SNS sodium channel fused to glutathione-S-transferase (GST). This may be immobilized on glutathione sepharose or agarose beads.

In an in vitro assay format of the type described above the putative inhibitor compound can be assayed by determining its ability to diminish the amount of labelled binding partner (e.g. the GFP-fusion described hereinafter) which binds to the immobilized GST-SNS sodium channel. This may be determined by fractionating the glutathione beads by SDS-polyacrylamide gel electrophoresis. Alternatively, the beads may be rinsed to remove unbound protein and the amount of protein which has bound can be determined by counting the amount of label present in, for example, a suitable scintillation counter.

Another assay format is dissociation enhanced lanthanide fluorescent immunoassay (DELFIA) (Ogata et al, 1992). This is a solid phase based system for measuring the interaction of two macromolecules. Typically one molecule (either VGSC or binding partner) is immobilised to the surface of a multi well plate and the other molecule is added in solution to this. Detection of the bound partner is achieved by using a label consisting of a chelate of a rare earth metal. This label can be directly attached to the interacting molecule or may be introduced to the complex via an antibody to the molecule or to the molecules epitope tag. Alternatively, the molecule may be attached to biotin and a streptavidin-rare earth chelate used as the label. The rare earth used in the label may be europium, samarium, terbium or dysprosium. After washing to remove unbound label, a detergent containing low pH buffer is added to dissociate the rare earth metal from the chelate. The highly fluorescent metal ions are then quantitated by time resolved fluorimetry. A number of labelled reagents are commercially available for this technique, including streptavidin, antibodies against glutathione-S-transferase and against hexahistidine.

In an alternative mode, the one of the two proteins may be labelled with a fluorescent donor moiety and the other labelled with an acceptor which is capable of reducing the emission from the donor. This allows an assay according to the invention to be conducted by fluorescence resonance energy transfer (FRET). In this mode, the fluorescence signal of the donor will be altered when the two proteins interact. The presence of a candidate modulator compound which modulates the interaction will increase or decrease the amount of unaltered fluorescence signal of the donor.

FRET is a technique known per se in the art and thus the precise donor and acceptor molecules and the means by which they are linked to the binding partner and a VGSC protein may be accomplished by reference to the literature.

The interaction between a VGSC and binding partner may also be measured by fluorescence polarisation. Typically, binding partners are obtained as isolated peptides through chemical synthesis or as recombinant peptides or as purified peptides from tissue or cell sources. Full length binding partners or fragments thereof may be employed in combination with VGSC peptides representing regions of the binding partner and VGSC molecules thought to be involved in the binding interaction.

Either of the two peptides in the assay is labelled with a suitable label, typically a fluorescent label. The fluorescent peptide is placed in a sample tube and monochromatic light is passed through a polarizing filter onto the sample tube. The fluorophore will be excited by the polarised light bundle and the emitted light is measured. The emitted light will be scattered in all directions, because of the rotational behaviour of the small peptide in solution. This rotational behaviour changes when the peptide interacts with its larger binding partner, resulting in retention of the polarisation and reduced scatter of the emitted light. Inhibitors will be screened by reading out the changes in rotational energy of the complex from the degree of polarisation of the emitted light.

Suitable fluorescent donor moieties are those capable of transferring fluorogenic energy to another fluorogenic molecule or part of a compound and include, but are not limited to, coumarins and related dyes such as fluoresceins, rhodols and rhodamines, resorufins, cyanine dyes, bimanes, acridines, isoindoles, dansyl dyes, aminophthalic hydrazines such as luminol and isoluminol derivatives, aminophthalimides, aminonaphthalimides, aminobenzofurans, aminoquinolines, dicyanohydroquinones, and europium and terbium complexes and related compounds.

Suitable acceptors include, but are not limited to, coumarins and related fiuorophores, xanthenes such as fluoresceins, rhodols and rhodamines, resorufins, cyanines, difluoroboradiazaindacenes, and phthalocyanines.

A preferred donor is fluorescein and preferred acceptors include rhodamine and carbocyanine. The isothiocyanate derivatives of these fluorescein and rhodamine, available from Aldrich Chemical Company Ltd, Gillingham, Dorset, UK, may be used to label the binding partner and ER For attachment of carbocyanine, see for example Guo et al, J. Biol. Chem., 270; 27562-8, 1995.

Rather than using fluorescence detection, it may be preferred in assay formats to detect labels and interactions using surface enhanced Raman spectroscopy (SERS), or surface enhanced resonance Raman spectroscopy (SERRS) (see e.g. WO 97/05280).

An alternative assay format is a Scintillation proximity assay (SPA, Amersham Biosciences, UK). SPA uses microscopic beads containing scintillant that can be stimulated to emit light. This stimulation event only occurs when radiolabelled molecules of interest are bound to the surface of the bead. Specific bead types may be produced with different coatings for specific applications including; receptor-ligand binding, enzyme assays, radioimmunoassays, protein-protein and protein-DNA interactions.

Modulators of Interaction

In a further aspect, the present invention provides peptide compounds, and processes for devising and producing such compounds, which are based on the portions of the VGSC and binding partner light chain which interact with each other e.g. the amino terminal as described in the Examples below.

Modulators which are putative inhibitor compounds can be derived from the binding partner and VGSC protein sequences. Peptide fragments of from 5 to 40 amino acids, for example from 6 to 10 amino acids from the region of the binding partner and VGSC which are responsible for the interaction between these proteins may be tested for their ability to disrupt this interaction. Antibodies directed to the site of interaction in either protein form a further class of putative inhibitor compounds. Candidate inhibitor antibodies may be characterised and their binding regions determined to provide single chain antibodies and fragments thereof which are responsible for disrupting the interaction between the binding partner and VGSC.

For the screening methods of the invention, any compounds may be used which may have an effect on VGSC functional expression. Such an effect may, for example, be mediated by a direct effect on the channel, or indirectly by blocking or preventing the interaction between a binding partner and the VGSC.

In one aspect, a compound for use in downregulating functional expression of a VGSC may be a compound which binds specifically to the VGSC and/or the binding partner. For example, such a compound may bind to the C-terminal region of PAPIN. A compound may bind to a region of the Nav 1.8 gene at amino acids 893-1148, 1420-1472 and/or 1724-1844 of SEQ ID NO: 2, or at an equivalent location in a variant sequence, and may thereby prevent binding by PAPIN, periaxin and/or HSPC025 respectively. A compound may therefore prevent binding between the VGSC and a binding partner and thereby prevent the enhancement of VGSC functional expression normally caused by the binding partner.

Compounds (putative VGSC modulators) which may be used may be natural or synthetic chemical compounds used in drug screening programmes. Extracts of plants which contain several characterised or uncharacterised components may also be used. In preferred embodiments the substances may be provided e.g. as the product of a combinatorial library such as are now well known in the art (see e.g. Newton (1997) Expert Opinion Therapeutic Patents, 7(10): 1183-1194). The amount of putative modulator compound which may be added to an assay of the invention will normally be determined by trial and error depending upon the type of compound used. Typically, from about 0.01 to 100 nM concentrations of putative modulator compound may be used, for example from 0.1 to 10 nM. Modulator compounds may be those which either agonise or antagonise the interaction. Antagonists (inhibitors) of the interaction are particularly desirable.

In a further aspect, the present invention provides peptide compounds, and processes for devising and producing such compounds, which are based on the portions of the VGSC and binding partners which interact with each other e.g. the regions described in the Examples below.

Modulators which are putative inhibitor compounds can be derived from the binding partner and VGSC protein sequences. Peptide fragments of from 5 to 40 amino acids, for example from 6 to 16 amino acids from the region of a binding partner or VGSC which are responsible for the interaction between these proteins may be tested for their ability to disrupt this interaction. For example, such peptides may be derived from the region of amino acids 893-1148, 1420-1472 or 1724-1844 of the rat Nav1.8 sodium channel as given in SEQ ID NO: 2, or from the C-terminal 120 amino acids of the rat PAPIN protein as given in SEQ ID NO: 6.

Antibodies directed to the site of interaction in either protein form a further class of putative inhibitor compounds. Candidate inhibitor antibodies may be characterised and their binding regions determined to provide single chain antibodies and fragments thereof which are responsible for disrupting the interaction between a binding partner and VGSC. A suitable antibody may bind to either the VGSC or the binding partner, and thereby prevent or block the interaction between these molecules.

Antibodies may be raised against specific epitopes of the VGSC or binding partner of the invention. For example, antibodies may be raised specifically against those regions, as described above, which are involved in the interaction between the VGSC and the binding partner.

For the purposes of this invention, the term “antibody”, unless specified to the contrary, includes fragments which bind a VGSC or binding partner of the invention. Such fragments include Fv, F(ab′) and F(ab′)₂ fragments, as well as single chain antibodies. Furthermore, the antibodies and fragment thereof may be chimeric antibodies, CDR-grafted antibodies or humanised antibodies.

Antibodies of the invention can be produced by any suitable method. Means for preparing and characterising antibodies are well known in the art, see for example Harlow and Lane (1988) “Antibodies: A Laboratory Manual”, Cold Spring Harbor Laboratory Press, Cold Spring Harbor, N.Y. For example, an antibody may be produced by raising antibody in a host animal against the whole polypeptide or a fragment thereof, for example an antigenic epitope thereof, herein after the “immunogen”.

A method for producing a polyclonal antibody comprises immunising a suitable host animal, for example an experimental animal, with the immunogen and isolating immunoglobulins from the animal's serum. The animal may therefore be inoculated with the immunogen, blood subsequently removed from the animal and the IgG fraction purified.

A method for producing a monoclonal antibody comprises immortalising cells which produce the desired antibody. Hybridoma cells may be produced by fusing spleen cells from an inoculated experimental animal with tumour cells (Kohler and Milstein (1975) Nature 256,495-497).

An immortalized cell producing the desired antibody may be selected by a conventional procedure. The hybridomas may be grown in culture or injected intraperitoneally for formation of ascites fluid or into the blood stream of an allogenic host or immunocompromised host. Human antibody may be prepared by in vitro immunisation of human lymphocytes, followed by transformation of the lymphocytes with Epstein-Barr virus.

For the production of both monoclonal and polyclonal antibodies, the experimental animal is suitably a goat, rabbit, rat or mouse. If desired, the immunogen may be administered as a conjugate in which the immunogen is coupled, for example via a side chain of one of the amino acid residues, to a suitable carrier. The carrier molecule is typically a physiologically acceptable carrier. The antibody obtained may be isolated and, if desired, purified.

An antibody, or other compound, “specifically binds” to a protein when it binds with preferential or high affinity to the protein for which it is specific but does substantially bind not bind or binds with only low affinity to other proteins. A variety of protocols for competitive binding or immunoradiometric assays to determine the specific binding capability of an antibody are well known in the art (see for example Maddox et al, J. Exp. Med. 158, 1211-1226, 1993). Such immunoassays typically involve the formation of complexes between the specific protein and its antibody and the measurement of complex formation.

In a further aspect, decreased functional expression of a VGSC may be achieved by inhibiting the expression from the VGSC gene. For example, down-regulation of expression of a target gene may be achieved using anti-sense technology or RNA interference.

In using anti-sense genes or partial gene sequences to down-regulate gene expression, a nucleotide sequence is placed under the control of a promoter in a “reverse orientation” such that transcription yields RNA which is complementary to normal mRNA transcribed from the “sense” strand of the target gene. See, for example, Smith et al, (1988) Nature 334, 724-726. Such methods would use a nucleotide sequence which is complementary to the coding sequence. Further options for down regulation of gene expression include the use of ribozymes, e.g. hammerhead ribozymes, which can catalyse the site-specific cleavage of RNA, such as mRNA (see e.g. Jaeger (1997) The new world of ribozymes Curr Opin Struct Biol 7:324-335, or Gibson & Shillitoe (1997) Ribozymes: their functions and strategies form their use Mol Biotechnol 7: 242-251.)

RNA interference is based on the use of small double stranded RNA (dsRNA) duplexes known as small interfering or silencing RNAs (siRNAs). Such molecules are capable of inhibiting the expression of a target gene that they share sequence identity or homology to. Typically, the dsRNA may be introduced into cells by techniques such as microinjection or transfection. Methods of RNA interference are described in, for example, Hannon (2002) Nature 418: 244-251 and Elbashir et al (2001) Nature 411: 494-498.

Specificity of Modulation

Where any of the methods of identifying modulators of the SNS sodium channel utilizes a cell-based system, such methods may further include the step of testing the viability of the cells in the assay e.g. by use of a lactate dehydrogenase assay kit (Sigma). This step may provide an indication of any interference by the test agent of vital cellular functions.

Therapeutic Compositions and their Use

As used hereafter the term “VGSC modulator” is intended to encompass any and all of the above modulator compounds which may be identified using any of the assays or design methods of the invention.

VGSC modulators as described above may be provided isolated and/or purified from their natural environment, in substantially pure or homogeneous form, or free or substantially free of other materials from their source or origin. Where used herein, the term “isolated” encompasses all of these possibilities. They may optionally be labelled or conjugated to other compounds.

VGSC modulators may be useful in the treatment or prophylaxis of a wide range of disorders.

The VGSC modulators can be formulated into pharmaceutical compositions. These compositions may comprise, in addition to one of the above substances, a pharmaceutically acceptable excipient, carrier, buffer, stabiliser or other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

For delayed release, the modulators may be included in a pharmaceutical composition for formulated for slow release, such as in microcapsules formed from biocompatible polymers or in liposomal carrier systems according to methods known in the art.

For continuous release of peptides, the peptide may be covalently conjugated to a water soluble polymer, such as a polylactide or biodegradable hydrogel derived from an amphipathic block copolymer, as described in U.S. Pat. No. 5,320,840. Collagen-based matrix implants, such as described in U.S. Pat. No. 5,024,841, are also useful for sustained delivery of peptide therapeutics. Also useful, particularly for subdermal slow-release delivery to perineural regions, is a composition that includes a biodegradable polymer that is self-curing and that forms an implant in situ, after delivery in liquid form. Such a composition is described, for example in U.S. Pat. No. 5,278,202.

Thus in a further aspect, the present invention provides a pharmaceutical composition comprising a VGSC modulator peptide-encoding nucleic acid molecule and its use in methods of therapy or diagnosis.

In a further aspect, the present invention provides a pharmaceutical composition comprising one or more VGSC modulators as defined above and its use in methods of therapy or diagnosis.

In further aspects, the present invention provides the above VGSC modulators and nucleic acid molecules for use in the preparation of medicaments for therapy.

In one aspect, the invention includes a method of producing analgesia in a mammalian subject, which method includes administering to the subject a VGSC modulator of the present invention. Modulators of the channel may prevent transmission of impulses along sensory neurons and thereby be useful in the treatment of acute, chronic or neuropathic pain.

Acute pain is temporary, generally lasting a few seconds or longer. Acute pain usually starts suddenly and is generally a signal of rapid-onset injury to the body or intense smooth muscle activity. Acute pain can rapidly evolve into chronic pain. Chronic pain generally occurs over a longer time period such as weeks, months or years.

The VGSC modulators of the invention may be used in the treatment or prevention of acute or chronic pain, or to prevent acute pain evolving into chronic pain. Treatment of pain is intended to include any level of relief from the symptoms of pain, from a decrease in the level of pain to complete loss of the pain. Prevention includes the prevention of the onset of pain, and the prevention of the worsening of pain, for example the worsening of pain symptoms or the progression from acute pain to chronic pain.

Examples of types of chronic pain which may be treated or prevented with the VGSC modulators of the present invention include osteoarthritis, rheumatoid arthritis, neuropathic pain, cancer pain, trigeminal neuralgia, primary and secondary hyperalgesia, inflammatory pain, nociceptive pain, tabes dorsalis, phantom limb pain, spinal cord injury pain, central pain, post-herpetic pain and HIV pain, noncardiac chest pain, irritable bowel syndrome and pain associated with bowel disorders.

In a further aspect there is provided a method of preventing progression of pain in a subject at risk for developing such pain, comprising administering to the subject a VGSC modulator of the present invention.

A composition may be administered alone or in combination with other treatments (e.g. treatments having analgesic effect such as NSAIDS), either simultaneously or sequentially, dependent upon the condition to be treated.

Peptides (for example such as those designed or discovered to inhibit the interaction of a binding partner and VGSC as described above) may preferably be administered by transdermal iontophoresis. One particularly useful means for delivering compound to perineural sites is transdermal delivery. This form of delivery can be effected according to methods known in the art. Generally, transdermal delivery involves the use of a transdermal “patch” which allows for slow delivery of compound to a selected skin region. Although such patches are generally used to provide systemic delivery of compound, in the context of the present invention, such site-directed delivery can be expected to provide increased concentration of compound in selected regions of neurite proliferation. Examples of transdermal patch delivery systems are provided by U.S. Pat. No. 4,655,766 (fluid-imbibing osmotically driven system), and U.S. Pat. No. 5,004,610 (rate controlled transdermal delivery system).

For transdermal delivery of peptides transdermal delivery may preferably be carried out using iontophoretic methods, such as described in U.S. Pat. No. 5,032,109 (electrolytic transdermal delivery system), and in U.S. Pat. No. 5,314,502 (electrically powered iontophoretic delivery device).

For transdermal delivery, it may be desirable to include permeation enhancing substances, such as fat soluble substances (e.g., aliphatic carboxylic acids, aliphatic alcohols), or water soluble substances (e.g., alkane polyols such as ethylene glycol, 1,3-propanediol, glycerol, propylene glycol, and the like). In addition, as described in U.S. Pat. No. 5,362,497, a “super water-absorbent resin” may be added to transdermal formulations to further enhance transdermal delivery. Examples of such resins include, but are not limited to, polyacrylates, saponified vinyl acetate-acrylic acid ester copolymers, cross-linked polyvinyl alcohol-maleic anhydride copolymers, saponified polyacrylonitrile graft polymers, starch acrylic acid graft polymers, and the like. Such formulations may be provided as occluded dressings to the region of interest, or may be provided in one or more of the transdermal patch configurations described above.

In yet another embodiment, the compound is administered by epidural injection. Membrane permeation enhancing means can include, for example, liposomal encapsulation of the peptide, addition of a surfactant to the composition, or addition of an ion-pairing agent. Also encompassed by the invention is a membrane permeability enhancing means that includes administering to the subject a hypertonic dosing solution effective to disrupt meningeal barriers.

The modulators can also be administered by slow infusion. This method is particularly useful, when administration is via the intrathecal or epidural routes mentioned above. Known in the art are a number of implantable or body-mountable pumps useful in delivering compound at a regulated rate. One such pump described in U.S. Pat. No. 4,619,652 is a body-mountable pump that can be used to deliver compound at a tonic flow rate or at periodic pulses. An injection site directly beneath the pump is provided to deliver compound to the area of need, for example, to the perineural region.

In other treatment methods, the modulators may be given orally or by nasal insufflation, according to methods known in the art. For administration of peptides, it may be desirable to incorporate such peptides into microcapsules suitable for oral or nasal delivery, according to methods known in the art.

Whether it is a peptide, antibody, nucleic acid molecule, small molecule or other pharmaceutically-useful compound according to the present invention that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

Instead of administering these agents directly, they could be produced in the target cells by expression from an encoding gene introduced into the cells, e.g. in a viral vector (a variant of the VDEPT technique—see below). The vector could be targeted to the specific cells to be treated, or it could contain regulatory elements which are switched on more or less selectively by the target cells.

Alternatively, the agent could be administered in a precursor form, for conversion to the active form by an activating agent produced in, or targeted to, the cells to be treated. This type of approach is sometimes known as ADEPT or VDEPT; the former involving targeting the activating agent to the cells by conjugation to a cell-specific antibody, while the latter involves producing the activating agent, e.g. an enzyme, in a vector by expression from encoding DNA in a viral vector (see for example, EP-A-415731 and WO90/07936).

The expression of a binding partner as hereinabove defined in an organism may be correlated with the functional expression of VGSC in the organism, and this correlation may form the basis of diagnosis of diseases related to inappropriate VGSC expression.

The invention will now be further described with reference to the following non-limiting Figures and Examples. Other embodiments of the invention will occur to those skilled in the art in the light of these. Any reference mentioned herein, inasmuch as it may be required to supplement the common general knowledge of the person skilled in the art in practicing the invention, is specifically incorporated herein by reference in its entirety.

### EXAMPLES

Materials and Methods

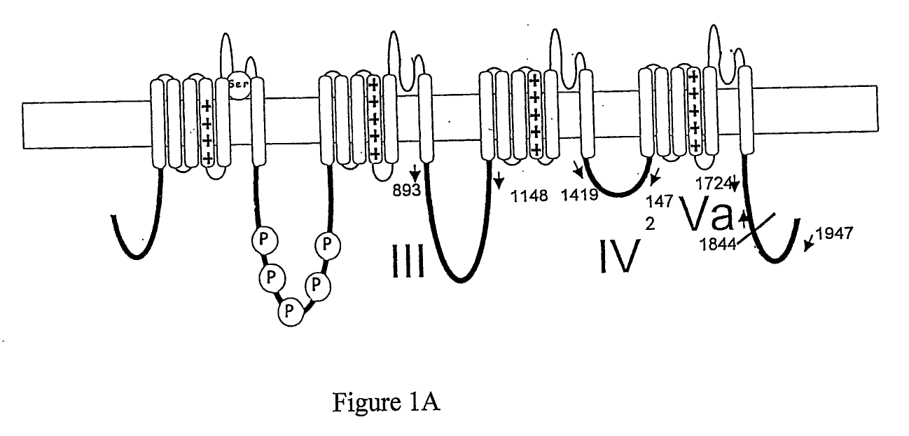

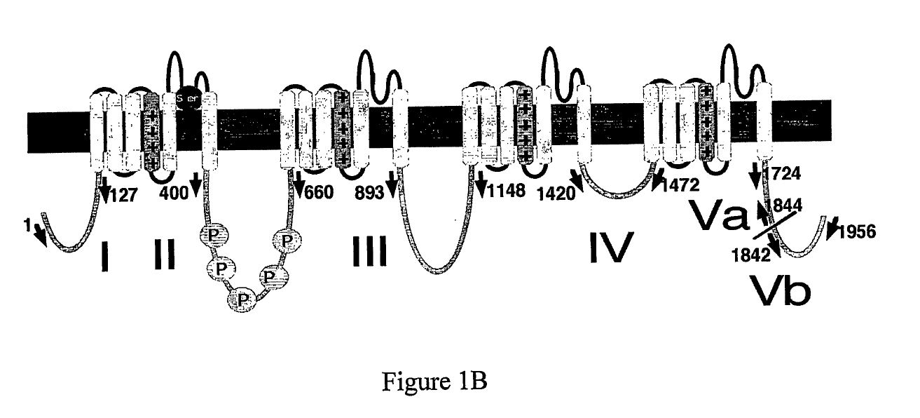

Using the yeast-2-hybrid system, proteins were identified that interact with the Nav1.8/SNS channel. The interaction trap was performed using the baits shown in FIG. 1 and fused to the DNA binding domain of LexA. For the baits the plasmids were generated with PCR using Nav1.8 as a template with different 5′ forward and 3′ reverse primers as detailed in hereinafter. The amplified fragments were ligated into pEG202 plasmid at EcoRI-NotI sites as an in-frame fusion with the LexA-DNA binding domain. This plasmid contains the selectable marker gene HIS3, and the plasmid containing this gene can be maintained in the yeast strain and selected on media lacking histidine. Yeast strain, EGY48, was transformed with the pEG202 containing the bait fragment/LexA. The binding sites for the bait/LexA were located upstream of 2 reporter genes. Firstly the upstream activating sequences of the chromosomal LEU2 gene, required in the biosynthetic pathway for leucine, were replaced in EGY48 with LexA operators, permitting selection for viability when cells were plated on media lacking leucine. This yeast strain also harbours a plasmid pSH18-34 that contains LacZ fusion gene, permitting discrimination based on colour and also contains the selectable marker gene URA3, allowing selection on media lacking uracil. The rat dorsal root ganglion (DRG) cDNA library was cloned in the plasmid pJG4-5 at EcoRI-XhoI sites and fused to transcription activation domain. This library containing plasmid also contained the selectable marker gene TRP1 allowing selection of library plasmids on media lacking tryptophan. The interaction trap was performed where the EGY48/pSH18-34 containing the bait plasmid pEG202 was transformed with the conditionally expressed rat DRG cDNA library in pJG4-5. Expression of library encoded proteins was induced by plating transformants on galactose/raffinose (Gal/Raf) plates lacking uracil (Ura-), histidine (His-), tryptophan (Trp-), and leucine (Leu-). In addition to the mutation in the LEU2 gene, EGY48 carries a mutation in three other marker genes (his3, trp1, ura3) that are needed for selection of the plasmids used in the interaction trap. The HIS3 gene carried by the bait plasmid pEG202 complemented the his3 mutation. The trp1 mutation was complemented by the library plasmid pGJ4-5 carrying the TRP1 gene and the ura3 mutation was complemented by the lacZ plasmid pSH18-34 containing the URA3 gene. So yeast cells containing library proteins that do not interact specifically with the bait protein will fail to grow in the absence of leucine. Yeast containing library proteins that interact with the bait will form colonies within 2 to 5 days on media lacking leucine, histidine, uracil and tryptophan and the colonies will turn blue as these colonies produce β-galactosidase when the reporter gene is transcribed and therefore turn blue on plates containing X-gal. The plasmids were isolated and characterised by a series of tests to confirm specificity of the interaction with the initial bait protein. Those found to be specific were then sequenced.

Plasmids and Yeast Strains:

pEG202:

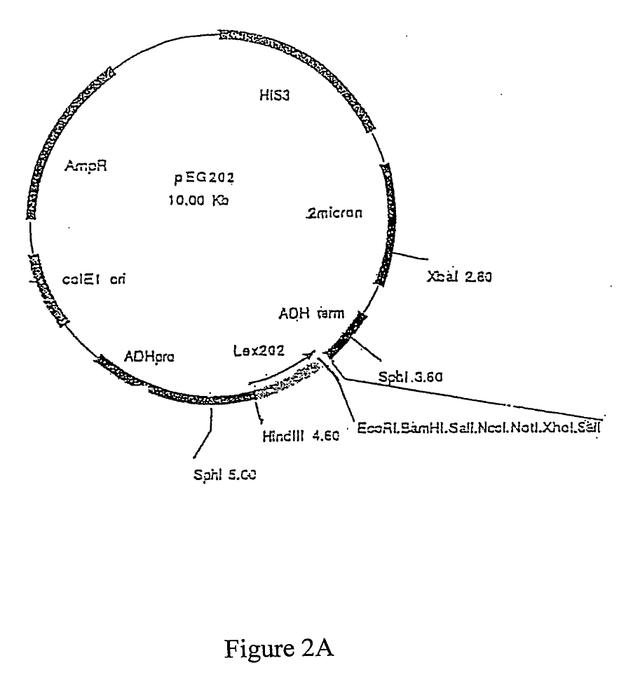

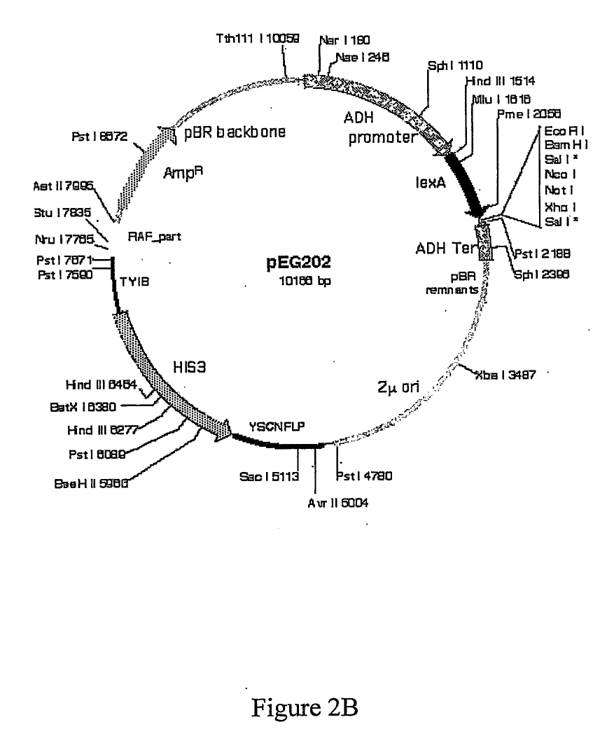

To make a plasmid that directs the synthesis of the bait proteins, the individual baits were inserted into pEG202 plasmid at EcoRI and NotI sites. FIG. 2 shows the map of the pEG202 plasmid. This plasmid is a yeast-E. coli shuttle vector and is a multi-copy plasmid containing the yeast 2 μm origin of replication. The plasmid also contains the selectable marker gene HIS3, along with yeast promoter ADH1 gene, followed by full length LexA coding region. This is followed by the ADH1 terminator sequences. Bait proteins expressed from this plasmid contain the amino acids 1-220 of the bacterial repressor protein LexA, which includes the DNA binding domain. The plasmid also contains the E. coli origin of replication and the ampicillin resistant gene. Downstream of the LexA coding region are unique restriction enzyme cloning sites EcoRI, BamHI, SalI NcoI, NotI and XhoI.

LEU2 Reporter Strain:

The interaction trap uses a yeast stain, EGY48 that has an integrated LEU2 gene with its upstream regulatory region replaced by LexA operators. This strain cannot grow in the absence of leucine unless the LexAop-LEU2 gene is transcribed. The LEU2 reporter is very sensitive which is due to the presence of three high affinity lexA operators positioned near the Leu2 transcription start. The operators are from the colE1gene and each can potentially bind two LexA dimers (Ebina et al, 1983 J Biol Chem 258: 13258-13261). The sensitivity of EGY48 can be of an advantage in isolating weak interactors, but it can also be too sensitive to use with baits that are themselves weak transcription activators. In addition to the mutation in the endogenous LEU2 gene, EGY48 carries mutations in three other marker genes, his3, trp1, ura3, that are needed to allow selection.

LacZ Reporter Plasmids:

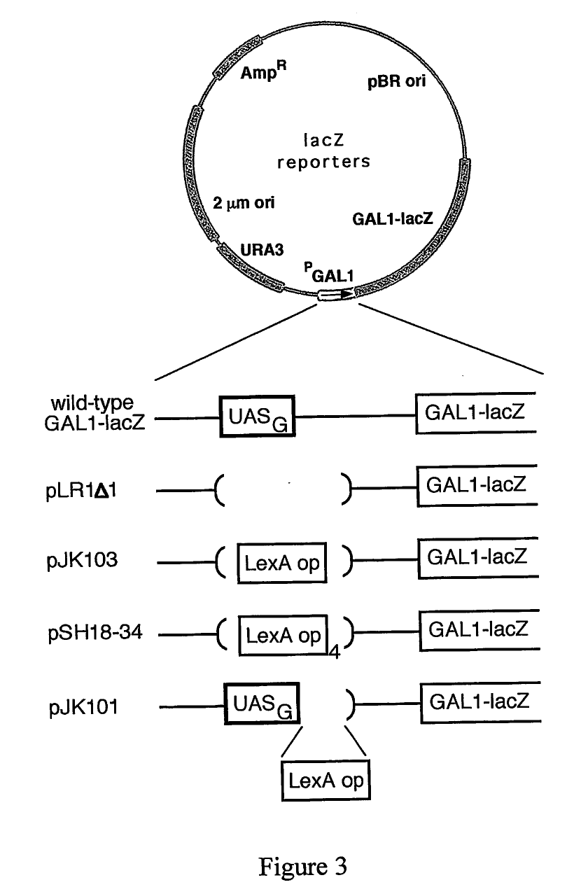

Reporters for measuring activation were derived from the pLR1Δ1 plasmid, in which the Gal1 upstream activating sequences (UASG) have been deleted. LexA operators have replaced the UASG. The LacZ reporter plasmid resides on the yeast origin of replication 2μ plasmids containing URA3 gene and the Gal1 TATA transcription start. It also contained the E. coli origin of replication and the ampicillin resistant gene. FIG. 3 shows in detail the various LacZ reporter plasmids. In the absence of interacting activation-tagged proteins, the yeast strain bearing these reporters do not make β-galactosidase and therefore appear white on X-Gal plates. Use of LacZ reporters provides two advantages as any false positive can be identified which may arise from activation of LEU2 reporter gene but which fail to activate the LacZ reporter. Secondly the LacZ reporters provides a relative measure of the amount of transcription caused by interaction of activation tagged cDNA protein with a bait as seen by a visual assay. The sensitivity of the LacZ reporters depends on the number of LexA operators positioned upstream of LacZ.

pSH18-34:

This plasmid was derived from the pLR1Δ1 plasmid where the UASG have been replaced by LexA operators and was used as a reporter gene to measure activation. The plasmid contained 4 of the high affinity overlapping type of colE1 LexA operator that can bind 4 LexA dimers and was more sensitive than plasmids which contain only 1 operator. This plasmid also contained the URA3 selectable marker gene.

pJK101:

This plasmid was used to measure repression by LexA fusions and was used as a positive control for the repression assay as it has the LacZ reporter insert. It contained most of the UASG and one colE1 operator between UASG and the Gal1 TATA transcription start which can bind 2 LexA dimers. The plasmid also contained the selectable marker URA3 gene.

pSH17-4:

This was a HIS3 2 μm plasmid encoding LexA fused to the activation domain of the yeast activator protein Gal4. This fusion protein strongly activates transcription and was used as a positive control in the activation assay.

pRFHM-1:

This plasmid was a 2 μm plasmid encoding LexA fused to the N-terminus of the drosophila protein bicoid. This fusion protein has no ability to activate transcription and can be used as a negative control for the activation assay and a positive control for the repression assay. This plasmid contained the selectable marker gene HIS3.

pEG22:

This was derived from the plasmid pEG202, where a region was deleted from restriction enzyme SphI to SphI site that included the whole of LexA region. pEG202 on its own is not a good negative control as the peptide encoded by the uninterrupted polylinker sequences is itself capable of weakly activating transcription. Once the LexA region was deleted the resulting plasmid can be used as a negative control for the repression assay.

Characterisation of the Bait Protein:

The major requirements for the bait protein were that it should not actively be excluded from the yeast nucleus and was capable of entering the yeast nucleus and binding LexA operator sites. Secondly it should not activate transcription of the lexA operator-based reporter genes on its own prior to the transformation of the library i.e it must not grow on media lacking leucine and the colonies should appear white on medium containing X-gal. The protocol is described by Ausubel et al, (1999 Short Protocols in Molecular Biology, Fourth Edition, John Wiley & Sons New York).

Activation Assay:

The activation assay confirms that the bait proteins are not activating transcription on their own. The method is described in full by Ausubel et al, 1999. The yeast strain was transformed with the reporter plasmid (pSH18-34) and grown on glucose minus uracil (Glu Ura-) plates. Colonies were picked and grown in Glu Ura-medium and the bait plasmid (pEG202), positive control (pSH17-4) and negative control (pRFHM1) were transformed and the transformants grown on Glu Ura- His-plates. Colonies were picked and grown onto Glu Ura- His—Xgal plates to look for LacZ expression. Colonies were also grown in Glu Ura- His-medium and grown on Gal/Raf Ura- His- and Gal/Raf Ura- His- Leu-plates to see if the bait was activating the reporter plasmid on its own.

In the Gal/Raf Ura- His- plates the positive and negative control as well as the bait plasmid gave colonies that grew at the same rate as was expected. As described previously the baits were fused to the LexA operators in the plasmid pEG202. Baits are chosen based on the sequence of the SNS sodium channel receptor. Baits were PCR generated using rat Nav1.8 cDNA as a template with bait III corresponding to position 893-1148 and bait IV corresponding to position 1420-1472. The C-terminal region, bait V was from position 1724 to position 1947. There was no library transformation present hence the colonies were grown on plates that contain tryptophan and leucine in the media. This showed that the bait protein was not toxic and can enter the yeast and survive. In the Gal/Raf Ura- His- Leu- only the positive control grew as there was no library plasmid present to turn on activation and allow colonies to be grown in the absence of leucine hence the negative control and the bait plasmids were not able to grow. In this assay only the positive control produced blue colonies on the Glu Ura- His—Xgal plates. This was what was expected. The bait plasmids did not produce blue colonies as the baits are not activating the reporter gene on their own and therefore there was no β-galactosidase activity and hence the colonies remain white on X-gal plates. The results for the activation assay are shown in Table 3.

TABLE 3

| | | Neg- | | | |
|---|---|---|---|---|---|
| | Positive | ative | | | |
| | control | control | Bait III | Bait IV | Bait V_{A} |
| Glu Ura⁻ His⁻ Xgal | Blue | White | White | White | White |
| Gal/Raf Ura⁻ His⁻ | + | + | + | + | + |
| Gal/Raf Ura ⁻ His⁻ Leu⁻ | + | −− | −− | −− | + |

It was concluded that baits III and IV did not activate transcription prior to library transformation and therefore could be used in the interaction trap. However bait V was seen to produce colonies in the absence of leucine and this showed that bait V was causing activation of the reporter gene on its own prior to library transformation. The next step in this stage was to cleave bait V into two separate fragments by standard cloning procedures (Ausubel et al, 1999) and produce new fusion proteins in pEG202 and repeat the activation assay. Resulting bait Va did not activate transcription on its own and therefore was able to be used in the interaction trap.

Repression Assay:

For bait-LexA proteins that do not activate transcription, it was important to confirm that the fusion protein was actually being synthesised in the yeast and was binding to the LexA operators by doing a repression assay. The repression assay was based on the observation that LexA and non-activating LexA fusions can repress transcription of a yeast reporter gene that has 1 LexA operator in between UASG and the TATA box. As mentioned previously LacZ expression was induced by galactose and was detectable in the presence of glucose because the negative regulatory elements that normally keep the Gal1 repressed in glucose were absent. The method is described by Ausubel et al, 1999. The yeast strain was transformed with the reporter plasmid pJK101 and selected on Glu Ura- plates, colonies were picked and grown in Glu Ura-medium and the plasmids containing the bait (pEG202), positive (pRFHM1) control and the negative (pEG22) control were transformed into the medium. The transformants were plated onto Glu Ura- His-plates and grown for a few days. Colonies were picked and streaked onto Glu Ura- HIs-Xgal and Gal/Raf Ura- His-Xgal and grown at 30° C. Yeast lacking LexA will begin to turn blue on the Gal/Raf Ura- His-Xgal after one day and will appear light blue on Glu Ura- His-Xgal after 2-3 days. The repression assay is summarised and shown in Table 4.

TABLE 4

| | Positive control | Negative control | Bait |
|---|---|---|---|
| Gal/Raf Ura⁻ His⁻-Xgal | Has high β- | Represses β- | Represses β-gal but |
| 1 day | galactosidase | galactosidase | more slowly than |
| | activity and there | activity and | the negative control |
| | are blue colonies | colonies turn blue | and blue colonies |
| | after a few hours. | after 1 day | appear at a slower |
| | | | rate |
| Glu Ura⁻ His⁻-Xgal | LacZ expression | Colonies appear | More profound |
| 2-3 days | detected and | light blue after 2 or | repression |
| | colonies appear | more days | |
| | blue. | | |

The positive control has a high β-galactosidase activity and the colonies turn blue on media containing Gal/Raf in the presence of X-gal. This LacZ expression is detectable in the presence of glucose because negative regulatory elements that normally keep GAL1 completely repressed in glucose are not present. An inert bait that makes LexA fused proteins, enters the nucleus and binds the lexA operators will block activation from the UASG repressing the LacZ expression 2 to 20-fold in the presence of galactose. Yeast containing a bait that enters the nucleus and binds operators turn blue more slowly than yeast lacking LexA i.e. the negative control. Bait proteins that do not activate in the activation assay, and do repress in the repression assay, were good candidates for use in an interaction trap. All of our baits could be used as they were seen to repress the β-galactosidase activity in X-gal medium and the colonies appeared at a slower rate than the negative control.

Interactor Hunt:

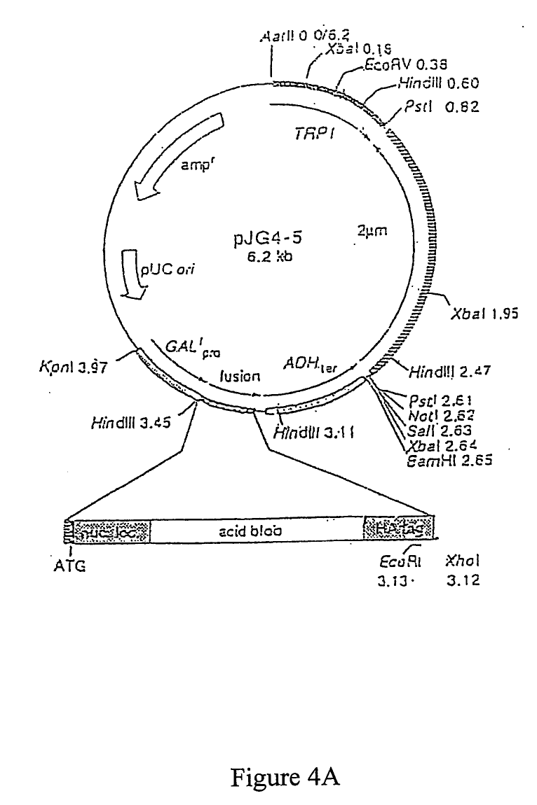

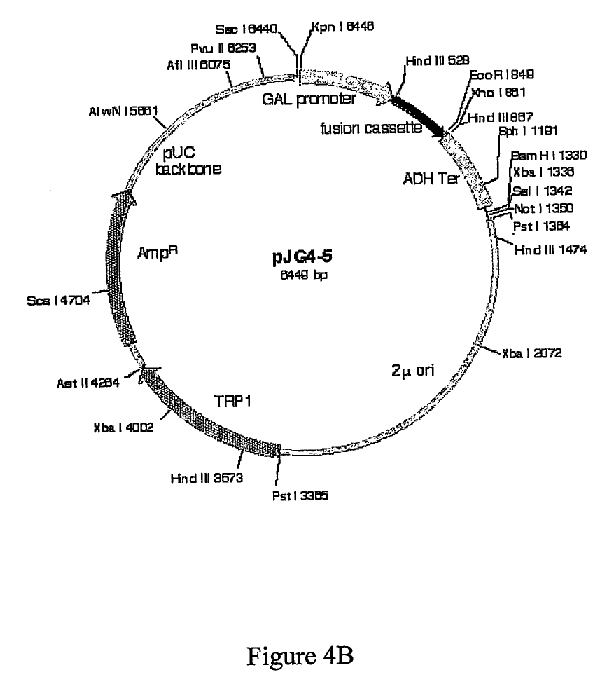

An interactor trap involved large platings of yeast containing LexA-fused baits, the reporter gene and the library in pJG4-5 with a cDNA expression cassette under the control of the GAL1 promoter as shown in FIG. 4. In the first plating, yeast was plated on complete minimal medium Glu Ura- His- Trp-dropout plates to select for the library plasmid. In the second plating, which selects for yeast that contains the interacting proteins, approximately 10⁶-10⁷ colonies were plated onto Gal/Raf Ura- His- Trp- Leu-dropout plates. Library plasmids from colonies identified in the second plating were purified by bacterial transformation and used to transform yeast cells for the final screen. Table 5 shows the final selection of a colony containing the library plasmid before bacterial miniprep was carried out to purify library containing plasmids and characterise them by sequencing.

TABLE 5
Positive colonies harbouring the library plasmid and showing an
interaction with the LexA-bait are chosen.

| 4 dish selection | Positive Colonies |
|---|---|
| Glu Ura⁻ His⁻ Trp^{- Xgal} | White |
| Gal/Raf Ura⁻ His⁻ Trp⁻ Xgal | Blue |
| Glu Ura⁻ His⁻ Trp⁻ Leu⁻ | −− |
| Gal/Raf Ura⁻ His⁻ Trp⁻ Leu⁻ | + |

Bait III

10⁶ cfu/10 cm dish were plated on Gal/Raf Ura- His- Trp- Leu-8 dishes were plated corresponding to 8×10⁶ cfu. 800 colonies were picked and plated for the 4 dish selection out of which 51 were blue on Gal/Raf Ura- His- Trp—Xgal.

Bait IV

10⁶ cfu/10 cm dish were plated on Gal/Raf Ura- His- Trp- Leu- and 8 dishes were plated corresponding to 10⁷ cfu. 1000 colonies were picked and plated for the 4 dish selection, out of which 10⁷ had blue colonies on Gal/RafUra- His- Trp—Xgal.

Bait V

This bait activated the LEU2 reporter gene on its own prior to library transformation therefore the bait was truncated into 2 separate fragments by designing primers and repeating the PCR to generate 2 separate fragments. The first fragment, Va was generated using forward and reverse primers and corresponded to amino acids position 1724 to 1844. Bait Va was plated at 10⁶ cfu/10 cm dish and 10 dishes were plated corresponding to 10⁷ cfu in total. 1000 colonies were streaked for each fragment and from the 4 dish selection, Va gave 27 blue colonies.

DNA sequencing was carried out on the positive colonies picked from the 4 dish selection to confirm what clone it was and also to eliminate duplicate sequences. In the final selection 39 clones were obtained with the interaction trap out of which 12 of the clones obtained were non-specific and for the final selection 27 positive clones were picked, of which 3 clones were unknown, that is they showed no homology to any known protein. The rest of the 24 clones isolated showed homology to known proteins. The results were tabulated and shown in Table 6.

TABLE 6
Positive clones as identified by DNA sequencing.

| Bait | Positive Clone |
|---|---|
| III-42 | Papin |
| IV-40 | Periaxin (myelinating protein) |
| Va-148 | HSPC025 (Unknown function) |

The clones identified and used in the following experiments were as follows:

PAPIN: the 201 amino acids of the C-terminal region were cloned by yeast-2-hybrid methods as described above. This clone was used in a GST-pull down assay and antisense experiment as described below.

Periaxin: the 482 amino acids of the C-terminal domain were cloned by yeast-2-hybrid methods as described above. This clone was used on GST-pull down assays and antisense experiments as decribed below.

HSPC025: A full length cDNA (1695 bp, 565 amino acids), including 21 bp 5′UTR and 178 bp 3′UTR was cloned by yeast-2-hybrid methods as described above. 1.4 kb from the N-terminal side including 21 bp of the 5′UTR was used in the antisense experiments described below. The full length cDNA including the 21 bp 5′UTR and 178 bp 3′UTR was used fro a GST-pull down assay and overexpression study in CHO-SNS22 cells, as described below.

Functional Experiments:

In Situ Hybridisation:

To determine whether the clones were expressed in Nav1.8-positive small diameter neurons in DRG, in situ hybridization was performed on 2 weeks old rat DRG sections. Clone III-42 (PAPIN) was excised out of the yeast expression vector pJG4-5 and sub-cloned into EcoRI and XhoI sites in pBluescript vector. Linearised III-42 DNA (EcoRI digested at 5′ end) was used to generate antisense from the 3′ to 5′ direction using T7 RNA polymerase and sense 5′ to 3′ probe using T3 polymerase. Digoxigenin-11-uridine-5′ triphosphate was used as a substrate for T7, T3 RNA polymerase to label RNA in in vitro transcription in place of UTP. Digoxigenin is linked to UTP via the C-5 position on the nucleotide. This Digoxigenin-labelled nucleotide can now be incorporated into nucleic acid probes RNA. A highly sensitive non-radioactive labelling and detection system based on the ELISA principle was used here. The DNA was modified with cardenolide-hapten digoxigenin (DIG) by enzymatic incorporation of digoxigenin labelled deoxyuridine-triphosphate (dUTP) with klenow enzyme. Following hybridisation of membrane with a digoxigenin labelled probe (DIG-labelled probe), the hybrids were detected by an ELISA reaction using DIG specific antibodies covalently coupled to the marker enzyme alkaline phosphatase. This binding of antibody:conjugated alkaline phosphatase was followed by an enzyme catalysed coupled redox reaction with the colour substrates 5-bromo-4-chloro-3-indolyl phosphate (BCIP) and nitroblue tetrazolium salt (NBT) which gives rise to a dark blue coloured water-insoluble precipitate directly adhering to the tissue. The sections were hybridised with the DIG-labelled probes overnight at 66° C. After washing the sections were visualised with alkaline phosphatase conjugated anti-digoxygenin antibody (Roche) and the sections viewed using the fluorescent microscope. The principle was that the DIG-labeled antisense mRNA probe will bind to the endogenous sense direction mRNA for III-42 as they have complementary sequences. An anti-Dig antibody conjugated to alkaline phosphatase will bind to the probe and this can be viewed in the microscope following a colourimetric reaction with the salts BCIP and NBT. Sense III-42 probe did not show any positive staining while antisense III-42 probe demonstrated strong staining in both small and large diameter neurons showing that III-42 is expressed in neurons that have endogenous Nav1.8. We also tested several other clones and they all showed expression in small diameter neurons.

Immunohistochemistry:

Immunohistochemistry studies were carried out to see if the protein of the clones isolated actually were expressed in the small diameter neurones. Cryosectioned tissues are fixed in paraformaldehyde and primary antibody applied followed by a secondary antibody and the sections viewed. Periaxin (IV-40) staining was seen both in the small diameter and the large diameter neurons. The periaxin antibody was a gift from Professor Peter Brophy (University of Edinburgh, UK). 1/1500 dilution of anti-L-Periaxin polyclonal antibody along with 1/10 dilution of anti-peripherin monoclonal antibody was applied to 2 weeks old sections of rat DRG. 1/200 dilution of secondary antibody, anti-rabbit IgG conjugated with FITC was used for periaxin and 1/50 dilution of anti-mouse IgG conjugated with texas red was used for peripherin. Fluorescese microscope was used with a blue filter to view the periaxin sections and a green filter to view the peripherin antibody. From the results it was seen that peripherin which acts as a positive control in this study was expressed in the small diameter neurones as expected. Periaxin has been shown to express in Schwann cells during myelination. We confirmed that periaxin was not expressed in axons but in the cells surrounding the axons i.e. Schwann cells. We also saw some periaxin staining in small and large diameter neurones. These results indicate that periaxin protein isolated in the yeast-hybrid system was actually expressed in neurones where Nav1.8 is expressed i.e. small diameter neurones.

Antisense:

To test the function of the clones on Nav1.8 in vivo, antisense was expressed in an expression vector in the 3′ to 5′ direction along with GFP and microinjected into nuclei of DRG neurons as described hereinafter. DNA sequencing was done to confirm the direction of the mRNA expression as well as to see the whether the correct expression vector was generated. The clones were microinjected individually. The principle of this method was that the generated 3′ to 5′ direction mRNA will bind to the endogenous sense direction mRNA for the corresponding clone and inhibit appropriate protein production. The list in Table 7 shows the total number of cells recorded for each pooled/individual antisense and the number of cells that did not exhibit Nav1.8/SNS current.

TABLE 7
Results of the different antisense microinjections into the nucleus of
cultured DRG neurones.

| | | | Mean current |
|---|---|---|---|
| | Number of cells | Number without | density/GFP |
| ANTISENSE | recorded | Nav 1.8 Current | current density |
| III-42 | 9 | 1 | 0.248 |
| VA-148 | 12 | 3 | 0.381 |
| IV-40 | 11 | 0 | 0.39 |

The mean peak sodium current is also shown and the last column measures the mean current density as compared to GFP mean current density. It can be seen that all 3 clones show significant effects on channel expression, as the presence of antisense oligonucleotides down regulates functional Nav1.8 expression.

Electrophysiology:

A stably transformed CHO cell line (CHO-SNS22 cells) that expresses rat Nav1.8 protein in the cytosol was transfected with the cDNA vector GFP-A148; (including the HSPC025 clone A148) by lipofection. CHO-SNS 22 cells are stably transfected cell line with rat SNS sodium channel cDNA. They do not have SNS sodium channel current however they express high amount of full length SNS sodium channel mRNA.

The CHO-SNS22 cell line was kept in Nutrient Mixture F-12 (Ham) medium (GibcoBRL) With 2.5% fetal bovine serum and 1 mg/ml Geneticin G418 sulphate. One day prior to transfection, cells were subcultured and plated in 35 mm dish containing F-12 medium with 0.5% fetal bovine serum and 1 mg/ml G418. Prior to transfection, cells in 35 mm dish were rinsed twice with serum-free F-12 medium. 1.1 μg of DNA was mixed with 5 μl of Lipofectamine (GibcoBRL) and incubated at room temperature for 30 min. The mixture was added to the pre-rinsed cells and incubated at 37° C. for 2 hours. DNA/lipofectamine mixture was replaced with F-12 medium with 0.5% fetal bovine serum and 1 mg/ml G418 after 2 hours.

Membrane currents were recorded from CHO-SNS 22 cells using the whole-cell patch-clamp technique. The extracellular recording solution contained the following (in mM): NaCl (140), TEA Cl (10) HEPES (10), CaCl₂ (2.1), MgCl₂ (2.12), 4-aminopyridine (4-AP) (0.5), KCl (7.5), tetrodotoxin (TTX) (250 nM). The solution was buffered to pH 7.2-3 with the addition of NaOH. The intracellular solution contained the following (in mM): CsCl (145), EGTA Na (3), HEPES (10), CaCl₂, (1.21), MgCl₂ (1.21), TEA Cl (10) and was buffered to pH 7.2-3 with the addition of CsOH. For recordings from neurons the extracellular solution was the same, except that NaCl was reduced to 43.3 mM with equivalent replacement of TEA-Cl and the addition of 20 μM CdCl₂. In the intracellular recording solution, 10% of the CsCl was replaced by CsF, the MgCl₂ replaced by 3 mM ATP (Mg) and the solution also contained 500 μM GTP (Li). Chemicals were either ‘AnalaR’ (BDH, Merk Ltd.) or supplied by Sigma. Chemicals were either ‘AnalaR’ (BDH, Merk Ltd., Lutterworth, Leicestershire, UK.), or supplied by Sigma (Poole, Dorset, UK). TTX was obtained from Alomone labs (TCS Biologicals, Botolph Claydon, Bucks, UK). A minority of CHO-SNS 22 cells generate an endogenous tetrodotoxin-sensitive (TTX-s) Na⁺ current (personal observation) which was eliminated from all recordings by including 250 nM TTX in the extracellular media No inward currents were recorded in non-transfected cells under these circumstances.

Electrodes were fabricated from thin-wall glass capillaries (GC150TF-10; Harvard apparatus, Edenbridge, Kent, UK), and had an access resistance of 2-3 MΩ when filled with recording solution. Recordings were made using an Axopatch 200B patch-clamp amplifier (Axon Instruments, Foster City, Calif., USA). Pulse protocols were generated and data stored to disk using pClamp6 software (Axon Instruments), running on a PC. CHO-SNS 22 cells were held at −90 mV. Voltage-clamp protocols incorporated a negative pre-pulse to −110 mV, and the cell was subsequently stepped to more depolarized potentials for 50 ms (up to a final value of +80 mV), in 10 mV increments.

All experiments were performed at room temperature.

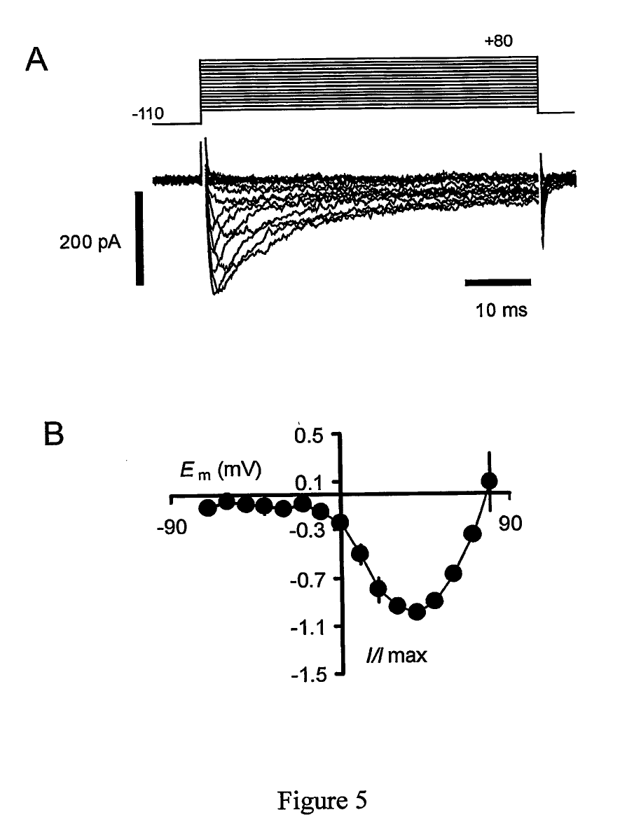

In 4 from a total of 22 CHO-SNS 22 cells transfected with the GFP-A48 full length clone, TTX-resistant (TTX-r) inward currents were recorded (FIG. 5). The current had characteristics of a Nav1.8 sodium current expressed in a heterologous system, and could not be distinguished from the current enabled by p11, that is known to be a sodium current.

In control GFP-only transfected cells, 1 in 43 cells generated a current (P=0.041, Fisher exact test), implying that A148 can contribute to the functional expression of Nav1.8.

Discussion:

The yeast two-hybrid system takes advantage of eukaryotic transcriptional activators which have two discrete molecular domains, a DNA binding domain and a transcriptional activation domain that can be exchanged from one transcription factor to another and still retain function. The DNA binding domain binds to a specific promoter sequence and the transcriptional activation domain directs the RNA polymerase II complex to transcribe the downstream gene. There are several variations of yeast two-hybrid systems which can be distinguished by their utilization of each domains. Fields and Song (1989 Nature 340: 245-246) first demonstrated the use of transcription factors when they reported protein-protein interactions by showing the interaction of two proteins if one was fused to the DNA binding domain and the other to an activation domain. They used yeast transcription factor Gal4 for both the DNA binding domain and transcriptional activation domain. Because of its strong transcriptional activity and endogenous expression of Gal4 in yeast, this method gives high sensitivity with high background. Gyuris et al. (1993 Cell 75: 791-803) modified this method altering Gal4 DNA binding domain to the bacterial repressor LexA and Gal4 transcriptional activation domain to bacterial activation domain B42. This was based on the system developed by Ma and Ptashne (1987 Cell 51: 113-119) where they generated a new class of yeast activators (B42) encoding E. coli genomic DNA fragments fused to the coding sequence of the DNA-binding domain of Gal4. They also generated a LexA fusion protein containing the new class of activating sequences fused to the DNA-binding domain of LexA. The acid blob B42 has relatively weaker transcriptional activity compare to Gal4 activation domain. Due to its bacterial origin, no endogenous yeast proteins bind to the LexA operators hence giving a system with low sensitivity. In addition to Gal4 and B42, the Herpes simplex virus protein VP16 is also used as a transcriptional activation domain in combination with Gal4 (Fearon et al., 1992 PNAS USA 89: 7958-7962) or LexA (Vojtek et al., 1993 Cell 74: 205-214) DNA binding domain, which does not have a nuclear localisation signal. The VP16 activation domain is fused to a nuclear localisation signal. Due to its higher transcriptional activity than Gal4 and B42, the systems which utilize VP16 are likely to have the highest sensitivity among the different yeast two-hybrid systems. In order to minimise the chance to clone non-specific interactor, we used the least sensitive system, LexA DNA binding domain and B42 transcriptional activation domain.

The sensitivity of the yeast two-hybrid systems also depends on reporters. Most systems use two reporter genes, one for enzymes required for the biosynthesis of an amino acid such as HIS3, LEU2 or URA3 genes and the other for enzymes which produce colour such as LacZ or CAT (chloramphenicol acetyl transferase). Using selectable markers for growth on a particular media has marked advantages of providing a selection for cDNA that encode interacting proteins rather than a visual assay which produce coloured colonies. The intensity of the expression of each reporter gene depends on the number of operators on the promoter region. The yeast strain we used, EGY48, has an integrated LEU2 gene with its upstream regulatory region replaced by six LexA operators. This was a very sensitive assay and can be activated by weak transcription activators fused to LexA. In our case we found this to be happening with bait V, so we truncated bait V into two separate fragments. For a second reporter, we chose the plasmid pSH18-34 as this has eight LexA operators positioned upstream of LacZ as compared to other plasmids such as pJK103 and pRB1840 which only have two and one LexA operator respectively. The advantage of using two reporter genes was to rule out possible false positives which can arise by activation of Leu2 gene by binding of weak activators to Leu2 promoters. These false positives can be identified as they will fail to activate the LacZ reporter. This means our system utilized the most sensitive reporter system driven by least sensitive DNA binding domain/transcriptional activation domain complex.

As described above, PAPIN is a member of a p120ctn family of proteins which have been identified as major substrates of tyrosine kinase phosphorylation enriched at adherens junctions (Reynolds et al, 1992 Oncogene 7: 2439-2445). NPRAP/δ-catenin also interacts with E-cadherin and β-catenin (Lu et al, 2002 J Neurosci Res 67(5): 618-624). PAPIN has 6 PDZ domains and may act as a scaffolding protein connecting components of epithelial junctions with p0071. The exact function of NPRAP/δ-catenin and p0071 is not known but since they are localised at cell-cell junctions suggests they may play a role as components of cell-cell junctions like p120ctn. So far there has been three reports for the interactions of PDZ domain-containing proteins and armadillo repeat-containing proteins. Adenomatous poyposis coli gene product interacts with PSD-95/SAP90 and SAP97/human discs-large tumour repressor gene (Matsumine et al, 1996 Science 272: 1020-1023). NPRAP/δ-catenin interacts with synaptic scaffolding molecule (Ide et al, 1999 Biochem Biophy Res Comm 256: 456-461) and NPRAP/δ-catenin and p0071 bind to PAPIN. As both the PDZ containing proteins and the armadillo repeat containing protein are localised at cell-cell junctions, their interaction may be important for the maintenance of the cell-cell junctions. Our isolated clone for PAPIN only had the last 210aa which contained the 2 PDZ domain in the C terminal of PAPIN and it is likely that Nav1.8 binds to this region.

Inflammatory pain that is characterised by a decrease in mechanical nociception threshold (hyperalgesia) arises through actions of inflammatory mediators. Hyperalgesia can occur through two pathways involving protein kinases. England et al (1996 J Physiol 495 (Pt 2) 429-440) and Gold et al (1996 Neurosci Lett 212: 83-86) both independently showed that the inflammatory mediators prostaglandin E2 PGE2), serotonin and adenosine produce hyperalgesia through cAMP-dependent protein kinase A (PKA) phosphorylation of the TTXr channels. Cesare et al, 1999 (Neuron 23 617-624) has shown that bradykinin induced sensitisation of nociceptive heat receptors is through protein kinase C (PKC). PKA and PKC mediate nociceptive sensitisation by modulating the activity of TTXr sodium currents (Gold et al, 1996).

Okuse et al, (1997 Mol Cell Neurosci 10: 196-207) investigated the expression of Nav1.8 in inflammatory and neuropathic pain models. They investigated the level of mRNA Nav1.8 in DRG after treatment with inflammatory stimuli such as Freund's adjuvant which involves a range of inflammatory mediators or NGF which acts directly on sensory neurones to exert hyperalgesic effect (Lewin et al, 1994 Eur J Neurosci 6: 1903-1912). They found 72 hours after Freund's adjuvant was injected into the footpad there was no change in the expression of Nav1.8 mRNA in L4 and L5 DRG although there was profound hyperalgesia In the presence of NGF there was a small increase in membrane associated Nav1.8 protein in DRG although the mRNA expression did not alter. They concluded that NGF was not necessary for the expression of Nav1.8 mRNA in experiments and Nav1.8 mRNA was not up-regulated in peripheral inflammatory states. They also found that in neuropathic states such as spinal nerve ligature and streptozotocin diabetic rat that leads to allodynia there was a down regulation of Nav1.8 mRNA levels. They concluded that Nav1.8 was not necessary for development of allodynia.

Schwann cells primary function is to myelinate nerve fibres and to promote rapid nerve impulse transmission, but it has also got a role in providing trophic support for spinal motorneurones and DRG neurones. Periaxin was first identified as a protein of myelinating Schwann cells in a screen for novel cytoskeleton-associated proteins with a role in peripheral nerve myelination (Gillespie et al, 1994 Neuron 12, 497-508). Like PO, the major integral membrane protein of peripheral nervous system myelin, periaxin is detectable at early stages of peripheral nervous system development (Scherer et al, 1995 Development 121: 4265-4273). The developmentally regulated nucleocytoplasmic redistribution of L-periaxin in embryonic Schwann cells is the first such example for a PDZ domain protein. Data have suggested that the nucleocytoplasmic distribution of several proteins that undergo active nuclear uptake is affected by cell-cell contact (Pedraza et al, 1997 Neuron 18: 579-589). The appearance of appropriate binding partners at the cell surface of Schwann cells may be the stimulus for the translocation of L-periaxin from the nucleus to myelinating processes as they ensheath the axon. Shermann et al, (2000, J Biol Chem 275: 4537-4540) suggest that nuclear targeting of L-periaxin in embryonic Schwann cells may sequester the PDZ domain from inappropriate interactions in the cytoplasm until the correct ligand becomes available at the cell cortex of the maturing myelin-forming Schwann cells. It has been shown that the stimulus that influences nuleocytoplasmic distribution is cell-cell contact (Gottardi et al, 1996 PNAS USA 93: 10779-10784), though zyxin, a LIM domain protein which also shuttles between the nucleus and the focal contacts, does so in response to cell-substrate interaction (Nix et al, 2001 J Biol Chem 276: 34759-34767). PDZ domains are known to be involved in protein-protein interaction but in our case we found that Nav1.8 does not bind to the PDZ domain of periaxin as our isolated clone did not contain this region. Further experiments have to be done to see which region of periaxin the Nav1.8 binds to. Studies carried out with periaxin gene knockout mice (Gillespie et al, 2000 Neuron 26: 523-531) have shown that mice assemble compact PNS myelin but it is unstable, leading to demyelination and reflex behaviours that are associated with the painful conditions caused by peripheral nerve damage. Older animals were seen to display extensive peripheral demyelination and a severe clinical phenotype with mechanical allodynia and thermal hyperalgesia which can be reversed by intrathecal administration of a selective NMDA receptor antagonist. Gillespie et al found that the when they examined the peripheral nerves of periaxin deficit mice to check whether the myelin sheath was affected, the demyelination was not apparent at 6 weeks. However at 6 months sensory, motor and autonomic nerves were extensively demyelinated. They found that saphenous nerves (sensory) were hypermyelinated but that C-fibres bundles that are unmyelinated were normal. The damage is confined to the myelin sheath and there was no difference seen in the number of L5 dorsal root ganglion between wild-type and periaxin deficient mice. Periaxin is one of a triplicate for the antisense expression vector microinjections that was seen to reduce the peak of the sodium current.

1-37. (canceled)

38. A method of identifying a modulator of a voltage gated sodium channel (VGSC), which method comprises:
(a) bringing into contact a test compound, a VGSC and one or more binding partners selected from the group consisting of PAPIN, periaxin and HSPC025 under conditions where the VGSC and the binding partner(s) are capable of forming a complex in the absence of the test compound; and
(b) measuring an activity of the VGSC, wherein a change in the activity of the VGSC relative to the activity in the absence of the test compound indicates that the test compound is a modulator of said VGSC.

39. A method according to claim 38 wherein said activity is the ability of the VGSC to form a complex with the binding partner(s).

40. A method according to claim 38 wherein said activity is the ability of the VGSC to mediate a sodium current across a membrane.

41. A method according to claim 38 wherein a decrease in the activity of the VGSC indicates that the test compound is an inhibitor of said VGSC.

42. A method according to claim 38 wherein said VGSC is a channel associated with responses to pain.

43. A method according to claim 38 wherein said channel is expressed in sensory neurons.

44. A method according to claim 43 wherein said channel is sensory neuron specific (SNS).

45. A method according to claim 38 wherein said channel is tetrodotoxin resistant.

46. A method according to claim 38 wherein said VGSC is selected from the group consisting of Nav 1.8, Nav 1.9 and Nav 1.3 sodium channels.

47. A method according to claim 38 wherein said VGSC has an amino acid sequence comprising:
(a) the Nav 1.8 amino acid sequence of SEQ ID NO:2 or SEQ ID NO:6,
(b) a species or allelic variant of (a),
(c) a variant of (a) having at least 70% amino acid sequence identity thereto, or
(d) a fragment of any of (a) to (c);
wherein said VGSC retains the ability to bind one or more binding partners selected from the group consisting of PAPIN, periaxin and HSPC025.

48. A method according to claim 47 wherein said VGSC retains the ability to mediate a sodium current across a membrane.

49. A method according to claim 38 wherein said PAPIN has an amino acid sequence comprising:
(a) the amino acid sequence of SEQ ID NO: 6,
(b) a species or allelic variant of (a),
(c) a variant of (a) having at least 70% amino acid identity thereto, or
(d) a fragment of any of (a) to (c);
wherein said PAPIN retains the ability to bind a VGSC.

50. A method according to claim 48 wherein said PAPIN has a sequence comprising amino acids 2566 to 2766 of SEQ ID NO:6.

51. A method according to claim 38 wherein said periaxin has an amino acid sequence comprising:
(a) the amino acid sequence of SEQ ID NO:8,
(b) a species or allelic variant of (a),
(c) a variant of (a) having at least 70% amino acid identity thereto, or
(d) a fragment of any of (a) to (c);
wherein said periaxin retains the ability to bind a VGSC.

52. A method according to claim 51 wherein said periaxin has a sequence comprising amino acids 902 to 1383 of SEQ ID NO:8.

53. A method according to claim 38 wherein said HSPC025 has an amino acid sequence comprising:
(e) the amino acid sequence of SEQ ID NO:10,
(f) a species or allelic variant of (a),
(g) a variant of (a) having at least 70% amino acid identity thereto, or
(h) a fragment of any of (a) to (c);
wherein said HSPC025 retains the ability to bind a VGSC.

54. A method according to claim 38 wherein at least one of said binding partner(s) is a full length binding partner protein or a species or allelic variant thereof.

55. A method according to claim 38 wherein said VGSC and said binding partner(s) are provided in a cell and said cell is contacted with a test compound.

56. A method according to claim 38 wherein said VGSC is provided in a cell in which the functional expression of said channel has been enhanced by increasing the level of one or more binding partners as defined in claim 38 in the cell.

57. A method according to claim 38 wherein said VGSC is provided in a cell which comprises a p11 peptide capable of binding said VGSC.

58. A method according to claim 38 comprising the steps of:
(i) providing a cell in which the functional activity of an SNS sodium channel has been enhanced by increasing the concentration of one or more of PAPIN, periaxin and HSPC025 in the cell;
(ii) contacting the channel in the cell with the test compound; and
(iii) measuring the activity of the channel.

59. A method according to claim 38 comprising the steps of:
(i) bringing into contact an SNS sodium channel, a binding partner selected from one or more of PAPIN, periaxin and HSPC025, and a putative modulator compound under conditions where the SNS sodium channel and the binding partner, in the absence of modulator, are capable of forming a complex; and
(ii) measuring the degree of inhibition of complex formation caused by said modulator compound.

60. A method according to claim 38 comprising the steps of:
(i) bringing into contact an SNS sodium channel, a binding partner selected from one or more of PAPIN, periaxin and HSPC025, and a putative modulator compound under conditions where the SNS sodium channel and the binding partner, in the absence of modulator, are capable of forming a complex;
(ii) exposing the SNS sodium channel to a stimulus such as to produce a sodium current across a membrane in which the SNS sodium channel is present; and
(iii) measuring the degree of inhibition of the current caused by said modulator compound.

61. A method according to claim 38 further comprising the step of formulating said test compound as a pharmaceutical composition.

62. A method according to claim 61 further comprising administering said formulation to an individual for the treatment of pain.

63. A compound identified by a method of claim 38.

64. A method of enhancing the functional expression of a voltage gated sodium channel (VGSC) in a cell which method comprises the step of increasing the level of one or more binding partner(s) as defined in claim 38.

65. A method according to claim 64 wherein said VGSC is a sensory neuron specific (SNS) sodium channel and wherein said binding partner(s) are one or more of PAPIN, periaxin and HSPC025.

66. A host cell capable of expressing a VGSC and a binding partner selected from one or more of PAPIN, periaxin and HSPC025 wherein said VGSC and/or said binding partner is expressed from one or more heterologous expression vectors within said cell.

67. A method of treating a disorder or condition associated with the activity of a voltage gated sodium channel, said method comprising administering to an individual in need thereof a compound according to claim 63 or an inhibitor of PAPIN, periaxin and/or HSPC025 activity or expression.

68. A method according to claim 67 wherein said treatment produces analgesia.

69. A method according to claim 67 wherein said treatment relieves chronic pain.

70. A method according to claim 67 wherein said compound is a antibody or fragment thereof specific to the PAPIN, periaxin and/or HSPC025 or antisense cDNA directed to the sequence encoding the PAPIN, periaxin and/or HSPC025.
